(19) United States
(12) Patent Application Publication
(10) Pub. No.: US 2022/0130513 A1
(43) Pub. Date: Apr. 28, 2022

(54) METHOD AND SYSTEM FOR DYNAMICALLY GENERATING PROFILE-SPECIFIC THERAPEUTIC PROTOCOLS USING MACHINE LEARNING MODELS

(71) Applicant: Mahana Therapeutics, Inc., San Francisco, CA (US)

(72) Inventor: Simon Levy, San Francisco, CA (US)

(73) Assignee: Mahana Therapeutics, Inc., San Francisco, CA (US)

(21) Appl. No.: 17/216,993

(22) Filed: Mar. 30, 2021

Related U.S. Application Data
(60) Provisional application No. 63/104,322, filed on Oct. 22, 2020.

(51) Int. Cl. G16H 20/70; G16H 50/70; G16H 50/20; G16H 40/67; G16H 10/60; A61B 5/00

(52) U.S. Cl. CPC G16H 20/70; G16H 50/70; A61B 5/4836; G16H 40/67; G16H 10/60; G16H 50/20

(57) ABSTRACT

A therapy is selected for administration to a patient, and the therapy is administered to the patient according to one or more therapeutic protocols. The patient's responses to the therapeutic protocols are monitored, collected, and correlated with the associated protocol data to generate data indicating the effectiveness of the protocols. The protocol effectiveness data is used as training data to train one or more machine learning based therapeutic protocol effectiveness prediction models. Data associated with one or more new protocols is provided as input to one or more of the trained therapeutic protocol effectiveness prediction models, which generates predicted protocol effectiveness data for the new protocols. The protocol effectiveness data is analyzed to determine and select one or more effective therapeutic protocols, resulting in generation of one or more maximally effective protocols.

### RELATED APPLICATIONS

This application claims the benefit of Paull et al., U.S. Provisional Patent Application No. 63/104,322, filed on Oct. 22, 2020, entitled “SYSTEMS AND METHODS FOR GUIDED ADMINISTRATION OF BEHAVIORAL THERAPY,” which is hereby incorporated by reference in its entirety as if it were fully set forth herein.

This application is related to U.S. patent application Ser. No. ______ (attorney docket number MAH009), naming Simon Levy as inventor, filed concurrently with the present application on Mar. 30, 2021, entitled “METHOD AND SYSTEM FOR DYNAMICALLY GENERATING GENERALIZED THERAPEUTIC PROTOCOLS USING MACHINE LEARNING MODELS,” which is hereby incorporated by reference in its entirety as if it were fully set forth herein. This application is also related to U.S. patent application Ser. No. ______ (attorney docket number MAH010), naming Simon Levy as inventor, filed concurrently with the present application on Mar. 30, 2021, entitled “METHOD AND SYSTEM FOR DYNAMICALLY GENERATING PROFILE-SPECIFIC THERAPEUTIC IMAGERY USING MACHINE LEARNING MODELS,” which is hereby incorporated by reference in its entirety as if it were fully set forth herein. This application is also related to U.S. patent application Ser. No. ______ (attorney docket number MAH011), naming Simon Levy as inventor, filed concurrently with the present application on Mar. 30, 2021, entitled “METHOD AND SYSTEM FOR DYNAMICALLY GENERATING GENERALIZED THERAPEUTIC IMAGERY USING MACHINE LEARNING MODELS,” which is hereby incorporated by reference in its entirety as if it were fully set forth herein.

### BACKGROUND

Every day, millions of people are diagnosed with a wide variety of medical conditions, ranging greatly in type and severity. A patient who has been diagnosed with a medical condition often experiences many hardships as a result of their diagnosis. In addition to physical effects, such as pain, discomfort, or loss of mobility that may accompany the diagnosis, the hardships faced by patients often further include financial difficulties resulting from lost work, medical bills and the cost of treatments. Further still, a patient's diagnosis often negatively impacts their social interactions, quality of life, and overall emotional well-being. The result is that many patients experience significant psychological distress, and often do not receive effective support or treatment to alleviate this distress.

Additionally, psychological distress often exacerbates the physical symptoms associated with a patient's diagnosis, which in turn can lead to even greater psychological distress. As one specific example, symptoms associated with a gastrointestinal (GI) disorder, such as irritable bowel syndrome (IBS), are often triggered by stress, and psychological issues such as depression and/or anxiety can worsen those symptoms. Often, when a patient is diagnosed with one or more medical conditions, the patient is referred to additional health practitioners for further care and treatment. For example, a patient who has been diagnosed with a gastrointestinal (GI) disorder may be referred to a psychologist, psychiatrist, counselor, or other mental health practitioner to address any psychological issues, such as stress, anxiety, and/or depression that may stem from the diagnosis. Health practitioners such as these typically utilize one or more techniques, methodologies, and/or modalities, such as, but not limited to, cognitive behavioral therapy (CBT), acceptance commitment therapy (ACT), dialectical behavioral therapy (DBT), exposure therapy, hypnotherapy, experiential therapy, and psychodynamic therapy, to assist patients with management of their physiological and/or psychological conditions.

Even though a behavioral therapy, such as CBT, can be implemented in a variety of ways, common elements can be identified. For example, CBT is typically administered utilizing a set of structured sessions or modules, which are each designed to teach a patient skills that enable the patient to better understand and manage thoughts and behaviors that may be negatively affecting their mental and physical states. Although aspects of CBT may be fairly structured, there is still enough flexibility to allow CBT to be adapted for use in treatment of particular conditions. One such example is that of utilizing CBT to treat IBS. The rationale for applying CBT to treat IBS is grounded in the biopsychosocial model, which states that one's biology, thoughts, emotions, external events, and behaviors influence IBS symptom expression in a bidirectional way.

A behavioral therapy, such as CBT, may be administered to patients across a range of delivery modalities. For example, the therapy may be administered by a health practitioner in-person, individually, or in-person, in a group. Alternatively, the therapy may be administered remotely, such as telephonically, over the internet, or through a computer software application. Further, a therapy may be administered to a patient without the direct involvement of a health practitioner, for example, a therapy may be administered to a patient remotely by a software application and/or may be self-administered by the patient. For any given therapy, regardless of delivery modality, one or more therapeutic protocols are typically defined for administration of the therapy, wherein the therapeutic protocols govern the manner in which the therapy is administered to a patient. For example, a therapy may include a series of lessons, questionnaires, and exercises, and a related protocol may dictate the order, speed, and/or frequency in which various lessons, exercises and questionnaires are presented to a patient. A protocol may also dictate the specific layout, content and general presentation of the various lessons, exercises and questionnaires. A protocol can be as specific as to dictate each word or sequence of words selected for use in the therapy. A therapy may be administered to a patient according to any number of protocols or any number of combinations of protocols.

Upon administration of a therapy to a patient according to a particular protocol or combination of protocols, data may be generated and/or collected regarding the effectiveness of the therapy and/or the associated protocols. Upon a determination that a particular protocol is not effective in treating a specific patient, group of patients, or medical condition, the protocol may be adjusted in an effort to find a more effective manner in which to administer the therapy. As one simplified example, a current therapeutic protocol may dictate that a series of questions should be asked to the patient in the order “A, B, C.” After administration of the therapy, it may be determined that the protocol of asking the questions in the order “A, B, C” was not an effective protocol, in which case the protocol might be adjusted to instead ask the questions in the order “C, B, A.”

Unfortunately, due to the vast quantity of data related to therapies, protocols, combinations of protocols, and associated protocol effectiveness data, the task of determining which protocols are effective and which are not, and further determining how to adjust the protocols to maximize effectiveness, becomes a monumental task. The problem is further compounded when you take into account that while certain protocols may be effective for one type of patient, the same protocols may not be effective for other types of patients, and so the protocols need to be tailored to particular patient characteristics in order to be maximally effective. The result is that there are thousands or millions of possible protocols or combinations of protocols that need to be analyzed in light of patient data associated with thousands or millions of patients, in order to provide the most effective treatment to patients. Clearly, this task is not feasible for a single human being or even a group of human beings to complete, even given unlimited time and resources.

Therefore, generation of effective therapeutic protocols presents a technical problem, which requires a technical solution. As software applications continue to replace human interactions, this problem becomes even more pronounced, as people are increasingly relying on applications to provide them with support and assistance in a wide variety of aspects of their daily lives. This is especially true in times of global crises, such as the 2020 worldwide pandemic, which has limited the availability and/or desirability of in-person medical appointments. When administering a therapy remotely, for example, over the interne, through a website, or through a software application, the protocols utilized are traditionally statically programmed into the software and thus are not able to be readily modified when new data, such as data relating to the effectiveness of the protocols, is received. Thus, due to the large number of people diagnosed with medical conditions, and the increasing demand for remote administration of therapies, the failure of traditional solutions to address the problem of dynamically generating therapeutic protocols, in order to reliably administer effective therapies to patients, has the potential to lead to significant consequences for a large number of people.

What is needed, therefore, is a technical solution to the technical problem of dynamically generating therapeutic protocols to ensure that patients receive effective care, support, and treatment.

### SUMMARY

Embodiments of the present disclosure provide a technical solution to the technical problem of dynamically generating therapeutic protocols to ensure that patients receive effective care, support, and treatment. In the disclosed embodiments, when a patient has been diagnosed with one or more health conditions, an appropriate therapy is selected for administration to the patient, depending on the particular diagnosis. In many instances, the therapy selected may be a psychological therapy that is intended to treat psychological issues related to the patient's diagnosis. As used herein the term “therapy,” “psychological therapy,” or “therapeutic modality” may include psychological techniques, methodologies, and/or modalities utilized to treat patients, such as, but not limited to psychotherapy, cognitive behavioral therapy (CBT), acceptance commitment therapy (ACT), dialectical behavioral therapy (DBT), exposure therapy, hypnotherapy, experiential therapy, and psychodynamic therapy.

In one embodiment, once an appropriate psychological therapy has been selected, the psychological therapy is administered to the patient according to one or more historical therapeutic protocols. As used herein, the phrase “administration of a therapy” may include administration of a therapy to a patient by a health practitioner, or administration of a therapy to a patient without the direct involvement of a health practitioner. As used herein, the term “protocol” or “therapeutic protocol” may include procedures and/or systems of rules for administration of a psychological therapy. A therapeutic protocol defines the rules, syntax, semantics, and synchronization of communications between a patient and the party that is administering the therapy. As noted above, a therapy being administered to a patient may include various lessons, exercises, and questionnaires. An associated therapeutic protocol, therefore, may dictate the order, speed, and/or frequency in which the various lessons, exercises and questionnaires are presented to a patient. A protocol may also dictate the specific content, layout, presentation, and word sequences selected for incorporation into the various lessons, exercises and questionnaires. As used herein, the term “historical therapeutic protocol” may include protocols that have previously been generated, tested, established, and/or clinically validated for use in administration of a therapy.

In one embodiment, as the psychological therapy is being administered to the patient, the patient's responses to the historical therapeutic protocols are monitored to obtain patient protocol response data. Patient protocol response data may also be collected after administration of the therapy. As used herein, in various embodiments, “patient response data” or “patient protocol response data” may include direct verbal or written feedback from the patient, indirect feedback, such as an indication of whether a particular therapeutic protocol appears to be having an effect on the patient, and/or other measureable data such as, but not limited to, physiological sensor data, and/or click-stream data showing patient engagement with the content of the therapy.

In one embodiment, the patient protocol response data is analyzed to determine the effectiveness of the therapeutic protocols administered to the patient as part of the therapy, and patient protocol effectiveness data is generated representing the effectiveness of the therapeutic protocols for the patient. In one embodiment, the patient protocol effectiveness data and patient data associated with the patient are analyzed to generate one or more patient profiles. As used herein, the term “patient data” may include data associated with the patient, such as, but not limited to age, sex, ethnicity, marital status, income level, geographic location, personal and family medical history, including the current medical issue that the therapy is designed to treat. As used herein, the term “patient profile” may include models or templates that describe a particular type of patient.

In one embodiment, historical therapeutic protocol data is correlated with patient profile data to generate therapeutic protocol effectiveness model training data, which is used as training data to train one or more machine learning based models. In one embodiment, the machine learning based models are models that predict the effectiveness of a given therapeutic protocol, and training the models with the therapeutic protocol effectiveness model training data results in the creation of one or more trained therapeutic protocol effectiveness prediction models. In various embodiments, the above described process may continue indefinitely, or may be terminated at any time at the discretion of an administrator of the method and system disclosed herein.

In various embodiments, once the therapeutic protocol effectiveness prediction models are trained, they can be used in a variety of ways. In one embodiment, the trained therapeutic protocol effectiveness prediction models can be used to dynamically generate an improved or maximally effective therapeutic protocol for a specific patient, or a specific type of patient. In other embodiments, the trained therapeutic protocol effectiveness prediction models can be utilized independently of a specific patient, for example, to generate an improved or maximally effective therapeutic protocol, which may be determined to be generally effective for patients, regardless of the patient's background and history.

In one embodiment, in the case of dynamically generating an improved or maximally effective therapeutic protocol for a specific patient, a psychological therapy is selected for administration to the patient. Patient data associated with the patient and patient profile data associated with the predefined patient profiles are analyzed to select a patient profile that is the best match for the specific patient. In one embodiment, the selected patient profile data is provided to the trained therapeutic protocol effectiveness prediction models.

In one embodiment, new therapeutic protocol test data, representing one or more new therapeutic protocols associated with the psychological therapy, is generated or otherwise obtained, wherein the new therapeutic protocols are new protocols to be considered for use in administration of the psychological therapy. In one embodiment, the new therapeutic protocol test data is provided to the trained therapeutic protocol effectiveness prediction models. In one embodiment, the trained therapeutic protocol effectiveness prediction models are utilized to generate predicted protocol effectiveness data for the new protocols represented by the new therapeutic protocol test data. In one embodiment, the predicted protocol effectiveness data associated with the new therapeutic protocols, and historical protocol effectiveness data associated with historical therapeutic protocols is analyzed to determine and select one or more effective therapeutic protocols.

In one embodiment, once one or more effective therapeutic protocols have been selected, effective protocol definition data associated with the one or more effective therapeutic protocols is utilized to generate one or more maximally effective therapeutic protocols for use in administration of the selected psychological therapy. In one embodiment, maximally effective protocol definition data associated with the one or more maximally effective therapeutic protocols is stored as historical protocol definition data for future use in administration of a psychological therapy. This allows the maximally effective therapeutic protocols to be later used in the generation of model training data, so that the therapeutic protocol effectiveness prediction models can be continually trained with new data. In one embodiment, the selected psychological therapy is then administered to the patient according to the maximally effective therapeutic protocols.

In one embodiment, in the case of generating an improved or maximally effective therapeutic protocol that is generally effective for patients, a process similar to that described above may be utilized, without providing patient-specific profile data to the trained therapeutic imagery effectiveness prediction models. For example, in some embodiments, a psychological therapy is selected for administration to one or more patients, new therapeutic protocol test data is generated and provided to the trained therapeutic protocol effectiveness prediction models, and the trained therapeutic protocol effectiveness prediction models are utilized to generate predicted protocol effectiveness data for the new protocols. In some embodiments, the predicted protocol effectiveness data and the historical protocol effectiveness data are analyzed to select one or more effective therapeutic protocols. Protocol definition data associated with the one or more effective therapeutic protocols is then utilized to generate one or more maximally effective therapeutic protocols. In one embodiment, maximally effective protocol definition data associated with the one or more maximally effective therapeutic protocols is stored as historical protocol definition data for future use in administration of a psychological therapy.

The above described processes result in generation of one or more maximally effective protocols, which may then be administered to a patient, thus ensuring that the patient receives effective care, support, and treatment. Further, the above machine learning process employs a feedback loop, such that the therapeutic effectiveness prediction models can be dynamically refined to account for newly received effectiveness data, thus improving the accuracy of the effectiveness predictions generated by the models.

As a result of these and other disclosed features, which are discussed in more detail below, the disclosed embodiments provide an effective and efficient technical solution to the technical problem of dynamically generating therapeutic protocols to ensure that patients receive effective care, support, and treatment.

### BRIEF DESCRIPTION OF THE DRAWINGS

FIG. 1A is a block diagram of a training environment for creating trained therapeutic protocol effectiveness prediction models, in accordance with one embodiment.

FIG. 1B and FIG. 1C are diagrams illustrating components of a therapy and the application of various therapeutic protocols to the therapy components, in accordance with one embodiment.

FIG. 2 is a block diagram of a runtime environment for utilizing trained therapeutic protocol effectiveness prediction models to generate maximally effective therapeutic protocols for a specific patient, in accordance with one embodiment.

FIG. 3 is a block diagram of a runtime environment for utilizing trained therapeutic protocol effectiveness prediction models to generate generalized maximally effective therapeutic protocols, in accordance with one embodiment.

FIG. 4 is a flow chart of a process for creating trained therapeutic protocol effectiveness prediction models, in accordance with one embodiment.

FIG. 5 is a flow chart of a process for utilizing trained therapeutic protocol effectiveness prediction models to generate maximally effective therapeutic protocols for a specific patient, in accordance with one embodiment.

FIG. 6 is a flow chart of a process for utilizing trained therapeutic protocol effectiveness prediction models to generate generalized maximally effective therapeutic protocols, in accordance with one embodiment.

Common reference numerals are used throughout the figures and the detailed description to indicate like elements. One skilled in the art will readily recognize that the above figures are merely illustrative examples and that other architectures, modes of operation, orders of operation, and elements/functions can be provided and implemented without departing from the characteristics and features of the invention, as set forth in the claims.

### DETAILED DESCRIPTION

Embodiments will now be discussed with reference to the accompanying figures, which depict one or more exemplary embodiments. Embodiments may be implemented in many different forms and should not be construed as limited to the embodiments set forth herein, shown in the figures, or described below. Rather, these exemplary embodiments are provided to allow a complete disclosure that conveys the principles of the invention, as set forth in the claims, to those of skill in the art.

### Term Definitions

As used herein, the term “patient” or “participant” may include an individual who has been diagnosed with one or more health conditions, an individual who is the recipient of a therapy in a clinical or non-clinical setting, and/or an individual who has not been diagnosed with a health condition, but is a recipient of a therapy in a clinical or non-clinical setting. Therefore, although the term “patient” will be used commonly throughout the enclosed specification, the term “participant” may also be used to indicate that applications of the methods and systems disclosed herein, used outside of a clinical setting, are also contemplated by the following disclosure.

As used herein the term “therapy,” “psychological therapy,” or “therapeutic modality” may include psychological techniques, methodologies, and/or modalities utilized to treat a patient, such as, but not limited to psychotherapy, cognitive behavioral therapy (CBT), acceptance commitment therapy (ACT), dialectical behavioral therapy (DBT), exposure therapy, hypnotherapy, experiential therapy, and psychodynamic therapy.

As used herein, the phrase “administration of a therapy” or “administering a therapy” may include providing, delivering, and/or applying a therapy to a patient. A therapy may be administered to a patient directly by a health practitioner. A therapy may be administered to a patient remotely, for example, over the internet or by computer software, without the direct involvement of a health practitioner. For example, the therapy may be self-administered by the patient. A therapy may also be administered to a patient remotely with partial involvement of a health practitioner. For example, the therapy to be administered may be selected by a health practitioner, but the therapy may then be self-administered by the patient, utilizing computer software, or the therapy may be administered to the patient by computer software, but a health practitioner may monitor the patient's response data.

As used herein, the term “protocol” or “therapeutic protocol” may include procedures and/or systems of rules for administration of a psychological therapy. A therapeutic protocol defines the rules, syntax, semantics, and synchronization of communications with a patient . For example, a therapy may include a series of lessons, questionnaires, and exercises, and a related protocol may dictate the order, speed, and/or frequency in which various lessons, exercises and questionnaires are presented to a patient. A protocol may also dictate the specific layout, content and general presentation of the various lessons, exercises and questionnaires. A protocol can be as specific as to dictate each word or sequence of words selected for use in the therapy. A therapy may be administered to a patient according to any number of protocols or any number of combinations of protocols.

As used herein, the terms “current therapeutic protocol” or “historical therapeutic protocol” may include protocols that have previously been generated, tested, established, and/or clinically validated for use in administration of a therapy.

As used herein, the terms “new protocol,” and “new therapeutic protocol” may include protocols that have not been previously generated, tested, established, and/or clinically validated for use in administration of a therapy. Additionally, the terms “new protocol,” and “new therapeutic protocol” may also include protocols that have been previously generated and/or tested, but may not yet be established and/or clinically validated for use in administration of a therapy. In various embodiments, new therapeutic protocols may also include potential therapeutic protocols or candidate therapeutic protocols, in the sense that they are protocols that are being considered for use in a therapy.

As used herein, the terms “improved protocol” or “improved therapeutic protocol” may include new therapeutic protocols that have been found to be more effective than a current or historical therapeutic protocol when used in a therapy to treat one or more patients, wherein effectiveness of a particular therapeutic protocol is determined by a variety of clinically validated outcome measures, as will be discussed in additional detail below.

As used herein, the terms “maximally effective protocol” or “maximally effective therapeutic protocol” may include therapeutic protocols that have been determined to be the most effective therapeutic protocols, for a particular period of time, out of the new, current, and/or historical therapeutic protocols of comparable type, wherein effectiveness of a particular therapeutic protocol is determined by a variety of clinically validated outcome measures, as will be discussed in additional detail below. An improved therapeutic protocol and/or a maximally effective therapeutic protocol may be the most effective, during the particular period of time, for the general patient population, the most effective for a particular group of patients, and/or the most effective for a specific individual patient, and the system and method disclosed herein accounts for these differences according to predefined guidelines, as will be discussed in further detail below.

As used herein, in various embodiments, the terms “patient response data” or “patient protocol response data” may include direct verbal or written feedback from the patient, indirect feedback, such as an indication of whether a particular therapeutic protocol appears to be having an effect on the patient, and/or other measureable data such as, but not limited to, physiological sensor data and/or click-stream data showing patient engagement with the content of the therapy.

As used herein, the term “patient data” may include data associated with a patient, such as, but not limited to age, sex, ethnicity, marital status, income level, geographic location, personal and family medical history, including the current medical issue that the therapy is designed to treat.

As used herein, the term “patient profile” may include models or templates that describe a particular type of patient.

### System

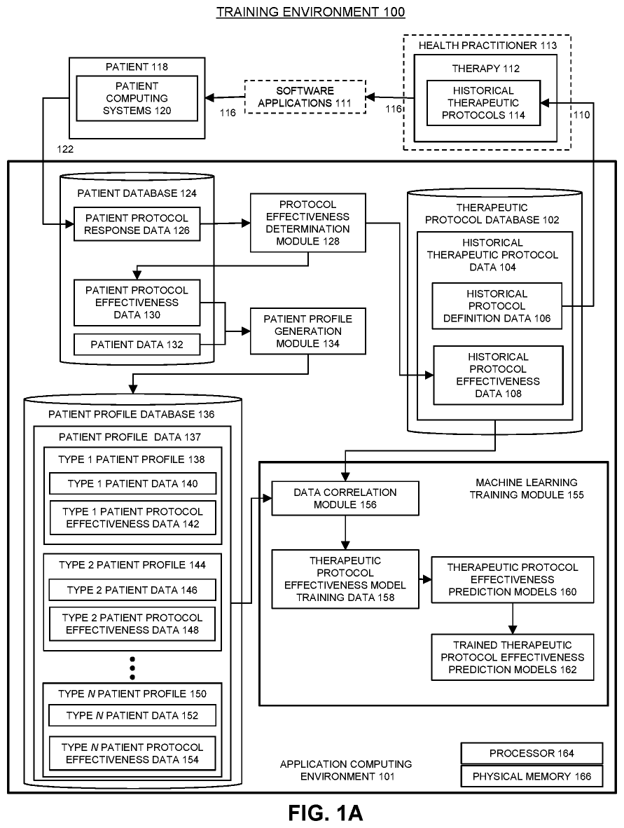

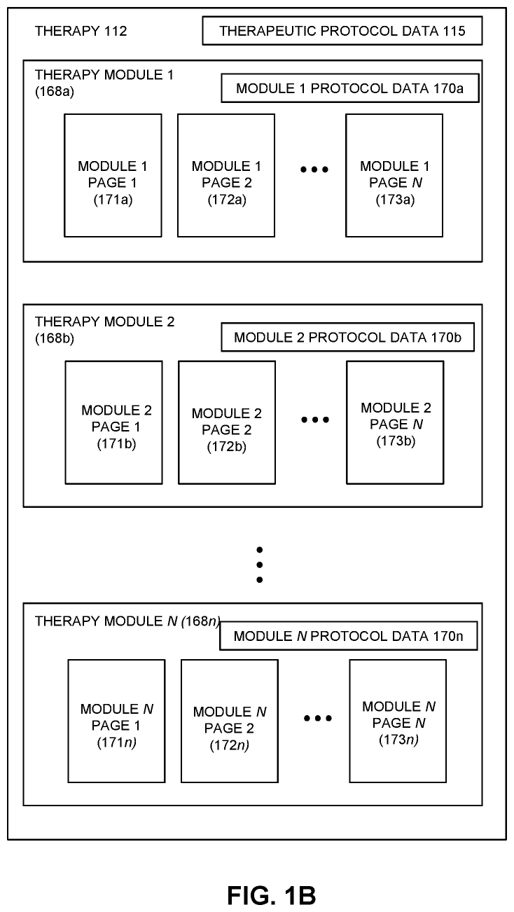

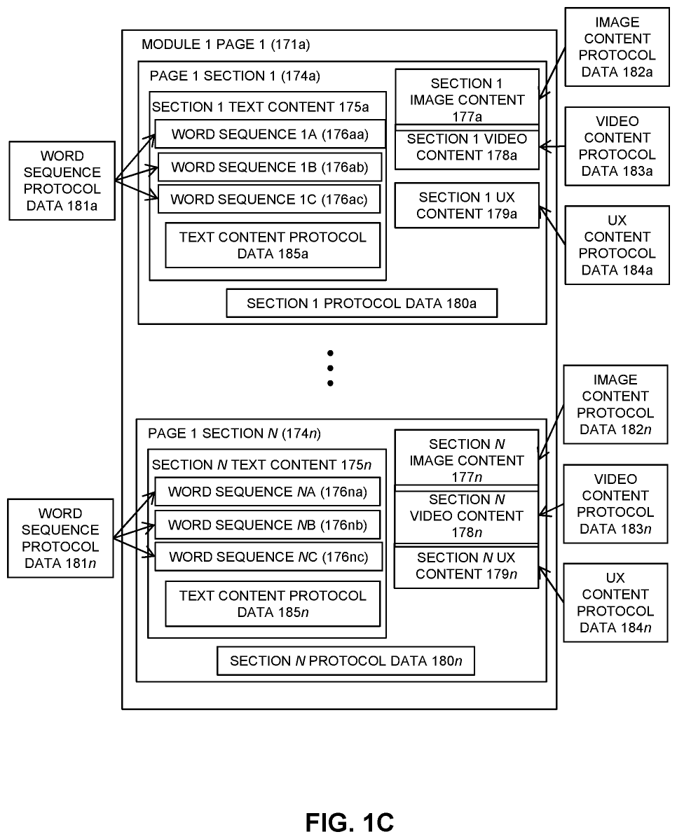

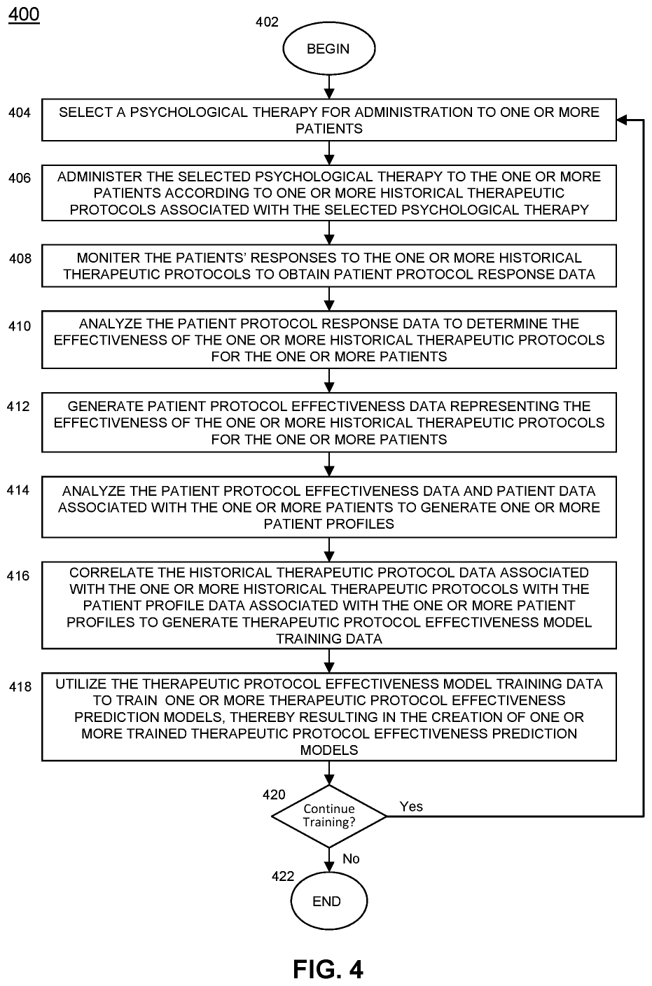

Embodiments of the present disclosure provide a technical solution to the technical problem of dynamically generating therapeutic protocols to ensure that patients receive effective care, support, and treatment. In the disclosed embodiments, an appropriate therapy is selected for administration to a patient, and the psychological therapy is administered to the patient according to one or more historical therapeutic protocols. In one embodiment, the patient's responses to the historical therapeutic protocols are monitored to obtain patient protocol response data, the patient protocol response data is analyzed to determine the effectiveness of the therapeutic protocols, and patient protocol effectiveness data is generated representing the effectiveness of the therapeutic protocols. In one embodiment, the patient protocol effectiveness data and patient data associated with the patient are analyzed to generate one or more patient profiles. In one embodiment, historical therapeutic protocol data is correlated with patient profile data to generate therapeutic protocol effectiveness model training data, which is used as training data to train one or more machine learning based models, resulting in the creation of one or more trained therapeutic protocol effectiveness prediction models. The above described system and process will be discussed in additional detail below with reference to the system of FIG. 1A and the process of FIG. 4.

FIG. 1A is a block diagram of a training environment 100 for creating trained therapeutic protocol effectiveness prediction models, in accordance with one embodiment.

In various embodiments, training environment 100 includes application computing environment 101, health practitioner 113, software applications 111, therapy 112 and associated historical therapeutic protocols 114, patient 118 and associated patient computing systems 120. In one embodiment, training environment 100 further includes communications channel 110, which facilitates retrieval of data from application computing environment 101 to be incorporated into therapy 112, communications mechanisms 116, which facilitates administration of therapy 112 to patient 118, and communications channel 122, which facilitates transmission of data from patient 118 to application computing environment 101. Each of the above listed elements will be discussed in further detail below.

In various embodiments, application computing environment 101 includes therapeutic protocol database 102, patient database 124, and patient profile database 136. In one embodiment, therapeutic protocol database 102 includes historical therapeutic protocol data 104, which further includes historical protocol definition data 106, and historical protocol effectiveness data 108. In one embodiment, patient database 124 includes patient protocol response data 126, patient protocol effectiveness data 130, and patient data 132. In one embodiment, patient profile database 136 includes patient profile data 137, which further includes type 1 patient profile 138, type 2 patient profile 144 through type n patient profile 150. In one embodiment, type 1 patient profile 138 further includes type 1 patient data 140 and type 1 patient protocol effectiveness data 142, type 2 patient profile 144 further includes type 2 patient data 146 and type 2 patient protocol effectiveness data 148, and type n patient profile 150 further includes type n patient data 152 and type n patient protocol effectiveness data 154. Each of the above listed elements will be discussed in further detail below.

In various embodiments, application computing environment 101 further includes several process modules, such as protocol effectiveness determination module 128, patient profile generation module 134, and machine learning training module 155. In one embodiment, machine learning training module 155 further includes data correlation module 156, therapeutic protocol effectiveness model training data 158, therapeutic protocol effectiveness prediction models 160, and trained therapeutic protocol effectiveness prediction models 162. In one embodiment, application computing environment 101 further includes processor 164 and physical memory 166, which together coordinate the operation and interaction of the data and data processing modules associated with application computing environment 101. Each of the above listed elements will be discussed in further detail below.

In one embodiment, patient 118 is a patient who has been diagnosed with a medical condition, and a determination is made as to whether patient 118 will benefit from receiving one or more therapies. In one embodiment, the determination is made by health practitioner 113. In one embodiment, the determination is made by patient 118. In one embodiment, the determination is made by computer software algorithms. In various other embodiments, the determination may be made by one or more third parties. As one specific example, symptoms associated with a gastrointestinal (GI) disorder, such as irritable bowel syndrome (IBS), are often triggered by stress, and psychological issues such as, but not limited to, stress, anxiety, and depression, can worsen those symptoms. Patients who are suffering from stress, anxiety, and/or depression related to their medical diagnosis often benefit from receiving certain types of psychological therapies to help them better understand and manage their physiological and/or psychological conditions. Examples of therapeutic modalities that may be beneficial to patients include, but are not limited to, cognitive behavioral therapy (CBT), acceptance commitment therapy (ACT), dialectical behavioral therapy (DBT), exposure therapy, hypnotherapy, experiential therapy, and psychodynamic therapy.

In one embodiment, once a determination has been made that patient 118 is likely to benefit from a particular therapy, such as therapy 112, the therapy 112 may be administered to the patient using one or more communication mechanisms 116. In some embodiments, communication mechanisms 116 include health practitioner 113 conducting a physical in-person meeting with patient 118 to verbally guide patient 118 through the therapy 112. In other embodiments, communication mechanisms 116 include administering the therapy 112 to patient 118 remotely, for example through a website, or through one or more software applications 111 that can be executed from patient computing systems 120. In one embodiment, the therapy 112 may be administered to patient 118 directly by health practitioner 113. In one embodiment, therapy 112 may be administered to patient 118 remotely, without the direct involvement of health practitioner 113. For example, therapy 112 may be self-administered by patient 118. In one embodiment, therapy 112 may also be administered to patient 118 remotely with partial involvement of health practitioner 113. For example, therapy 112 may be selected for administration by health practitioner 113, but therapy 112 may then be self-administered by patient 118, utilizing software applications 111, or therapy 112 may be administered to patient 118 by software applications 111, but health practitioner 113 may monitor patient 118's response data.

In various embodiments, patient computing systems 120 may include, but are not limited to, a desktop computing system, a mobile computing system, a virtual reality computing system, a gaming computing system, a computing system that utilizes one or more Internet of Things (IoT) devices, and/or any other type of computing system discussed herein, known at the time of filing, developed/made available after the time of filing, or any combination thereof

As noted above, there are a variety of established and/or clinically validated therapies that have been shown to provide benefit to patients, and administration of these clinically validated therapies are typically governed by a collection of therapeutic protocols associated with the particular therapy. As noted above, and as used herein, the term “protocol” or “therapeutic protocol” may include procedures and/or systems of rules for administration of a psychological therapy. A therapeutic protocol defines the rules, syntax, semantics, and synchronization of communications between a patient and the party that is administering the therapy. For example, a particular therapy being administered to a patient may include various modules that contain content such as lessons, exercises, and questionnaires. An associated therapeutic protocol, therefore, may dictate the order, speed, and/or frequency in which the various lessons, exercises and questionnaires are presented to a patient. A protocol may also dictate the specific layout, content and general presentation of the various lessons, exercises and questionnaires. A protocol can be as specific as to dictate each word or sequence of words selected for use in the therapy. For any given therapy, protocols can be defined and applied to the therapy as a whole, or to any individual component or sub-component of a therapy. A therapy may be administered to a patient according to any number of protocols or any number of combinations of protocols.

FIG. 1B and FIG. 1C are diagrams illustrating components of a therapy and the application of various therapeutic protocols to the therapy components, in accordance with one embodiment.

FIG. 1B is an illustrative example, according to one embodiment, depicting an overview of therapy components and associated protocols. In the illustrative example of FIG. 1B, therapy 112 is governed by associated therapeutic protocol data 115, which dictates the protocols to be applied to the entirety of therapy 112. For example, therapeutic protocol data 115 might include data such as, but not limited to, data indicating the number of modules in therapy 112, the order in which to present the modules to the patient, and the duration of time between the presentation of the modules to the patient. In the illustrative example of FIG. 1B, the therapeutic protocol data 115 indicates that therapy 112 should include N modules, and that the modules should be presented in sequential order from module 1 to module N. Therapeutic protocol data 115 may also dictate that each sequential module should be presented to the patient one week after the prior module has been completed.

As one specific example, administration of a cognitive behavioral therapy (CBT) is often divided into eight separate sessions, or modules, with each module being presented to the patient approximately one week apart from the previous module. As one example, a first module of a therapy, such as therapy module 1 (168a) of therapy 112, may be focused on providing education to the patient related to their medical condition and/or education regarding the therapy and its goals. A second module of a therapy, such as therapy module 2 (168b) of therapy 112, may involve having the patient complete a self-assessment regarding their thoughts, emotions, and behaviors, with the goal of helping the patient to develop an understanding of how the interaction between the patient's thoughts, emotions, and behaviors impact the patient's medical symptoms. A final module of a therapy, such as therapy module N (168n) of therapy 112, may be focused on helping the patient develop skills to assist in processing their emotions, developing long-term goals, and managing symptom flare-ups.

It should be noted that the above examples were given for illustrative purposes only and are not intended to limit the scope of the invention as disclosed and claimed herein. It should be apparent to one of skill in the art that any number of sessions or modules may be included as part of a therapy, and the goals of each module, the presentation sequence of the modules and/or any other aspect of the therapy may be dictated by the associated protocol data. As noted above, at a high level, therapeutic protocol data 115 governs therapy 112, and further, each of the modules present may be governed by individual module protocol data.

For example, in the illustrative embodiment of FIG. 1B, therapy module 1 (168a) is governed by module 1 protocol data 170a, therapy module 2 (168b) is governed by module 2 protocol data 170b, and therapy module N (168n) is governed by module N protocol data 170n. In various embodiments, the module protocol data dictates the protocols to be applied to the entirety of an individual module. In various embodiments, module protocol data might include data such as, but not limited to, data indicating the number of pages in the associated module, and the order in which to present the pages to the patient. Module protocol data may also contain textual data, such as the text that should make up the title of the associated module In the illustrative embodiment of FIG. 1B, the module 1 protocol data 170a indicates that therapy module 1 (168a) should include N pages, and that the pages should be presented in sequential order from page 1 to page N. In the illustrative example of FIG. 1B, therapy module 1 (168a) includes module 1 page 1 (171a), module 1 page 2 (172a), through module 1 page N (173a). Therapy module 2 (168b) includes module 2 page 1 (171b), module 2 page 2 (172b), through module 2 page N, (173b). Therapy module N (168n) includes module N page 1 (171n), module N page 2 (172n), through module N page N (173n).

It should be noted again that the examples given above are for illustrative purposes only and are not intended to limit the scope of the invention as disclosed and claimed herein.

FIG. 1C is an illustrative example, according to one embodiment, depicting a detailed view of individual therapy module page components and associated protocols. For example, in the illustrative example of FIG. 1C, Module 1 page 1 (171a) includes several sections, such as page 1 section 1 (174a) through page 1 section N (174n). Similar to the description above with respect to the individual modules, section protocol data such as section 1 protocol data 180a through section N protocol data 180n dictate the various protocols that govern the individual sections of a given page. In various embodiments, the section protocol data might dictate things such as, but not limited to, the number of sections on a given page, as well as the position, size, and general layout of the sections on the page.

Likewise, each page section may include different type of content, such as text content, image content, video content and user experience (UX) content. For example, in the illustrative embodiment of FIG. 1C, section 1 text content 175a and section N text content 175n are respectfully governed by text content protocol data 185a and text content protocol data 185n. Section 1 image content 177a and section N image content 177n are respectfully governed by image content protocol data 182a and image content protocol data 182n. Section 1 video content 178a and section N video content 178n are respectfully governed by video content protocol data 183a and video content protocol data 183n. Section 1 UX content 179a and section N UX content 179n are respectfully governed by UX content protocol data 184a and UX content protocol data 184n. Text protocol data, image content protocol data, video content protocol data, and UX content protocol data may govern things such as, but not limited to the content of the text, images, videos or UX elements that are present in each section of a given page.

Lastly, protocols can be defined on a very granular level, down to individual words or sequences of words. As illustrated in FIG. 1C, section 1 text content 175a may include a variety of word sequences, such as word sequence 1A (176aa), word sequence 1B (176ab), and word sequence 1C (176ac). Each of these word sequences may be governed by specific protocol data, such as word sequence protocol data 181a. Likewise, section N text content 175n may include a variety of word sequences, such as word sequence NA (176na), word sequence NB (176nb), and word sequence NC (176nc). Each of these word sequences may be governed by specific protocol data, such as word sequence protocol data 181n.

As can be seen from the illustrative embodiments of FIG. 1B and FIG. 1C, each individual therapy can be divided up into any number of sections and sub-sections, each with their own layer of protocols. Thus one therapy could contain thousands of individual protocols and there could be millions of ways of defining, combining, and arranging those protocols to create a protocol or combination of protocols that will be effective for use in administration of a given therapy. In the embodiments disclosed herein, the goal is the generation of improved protocols, or maximally effective protocols, wherein the maximally effective protocols are protocols that are the most effective, during a particular period of time, for the general patient population, the most effective for a particular group of patients, and/or the most effective for a specific individual patient.

Returning now to FIG. 1A, as will now be discussed in detail, the embodiments disclosed herein utilize machine learning models to generate improved and/or maximally effective therapeutic protocols for administration to one or more patients. In order to utilize machine learning models to generate improved and/or maximally effective therapeutic protocols, one or more therapeutic protocol effectiveness prediction models must first be trained, which is depicted in training environment 100 of FIG. 1A. Once therapy 112 has been selected for administration to patient 118, historical protocol definition data 106 is retrieved from therapeutic protocol database 102. As used herein, the term “historical therapeutic protocol” may include protocols that have previously been generated, tested, established, and/or clinically validated for use in administration of a therapy. Historical therapeutic protocol data 104 may include data such as historical protocol definition data 106, which defines one or more historical protocols. In the embodiments disclosed herein, it is expected that the number of historically defined protocols will be a very large number, due to the millions of ways of defining and combining those protocols, as noted above. In one embodiment, historical therapeutic protocol data 104 also includes historical protocol effectiveness data 108, which quantifies the effectiveness of each protocol or each combination of protocols represented by historical protocol definition data 106, as will be discussed in additional detail below.

In various embodiments, once the historical protocol definition data 106 is retrieved from therapeutic protocol database 102, a determination is made as to which historical therapeutic protocols 114 to use for administration of therapy 112 to patient 118. In one embodiment, this determination may be made at the discretion of health practitioner 113, who, in some embodiments, is the health practitioner responsible for the treatment of patient 118, and the determination may be based on a wide variety of factors, such as, but not limited to, the severity of the patient's symptoms, the patient's medical history, the patient's age group, the patient's sex, and the patient's ethnicity.

In one embodiment, once the selected therapy 112 is administered to patient 118 according to historical therapeutic protocols 114, the patient's responses to the therapy 112 and associated historical therapeutic protocols 114 are monitored to obtain patient protocol response data 126, which, in some embodiments, may then be stored in a data structure, such as patient database 124. As used herein, in various embodiments, “patient response data” or “patient protocol response data” may include feedback from the patient related to the historical therapeutic protocols 114 utilized in administration of the therapy 112. Patient protocol response data 126 may include direct verbal or written feedback, indirect feedback, such as an indication of whether a particular therapeutic protocol appears to be having an effect on the patient. The patient protocol response data 126 may further include any other measureable data such as, but not limited to, click-stream data showing details related to patient engagement with the content of the therapy, such as, but not limited to, the time that the patient spends engaging with each section of a particular therapy module. The patient protocol response data 126 may also include data received from devices such as, but not limited to, sleep trackers, or other types of physiological sensors that may be used to measure a patient's physiological state, such as heart rate, respiratory rate, and/or blood pressure. The patient protocol response data 126 may then be provided to protocol effectiveness determination module 128, which in one embodiment is responsible for analyzing the patient protocol response data 126 to determine and assign an effectiveness rating to one or more protocols or one or more combinations of protocols.

As one specific illustrative example, one session or module of the therapy 112 might be focused on learning to relax, improving sleep, and managing stress and emotions. A wide variety of data can be collected for classification as patient protocol response data 126. For example, health practitioner 113 may solicit direct feedback from the patient 118 after completion of the therapy session, such as in a verbal interaction with the patient, or through a survey or questionnaire administered in person or remotely, which asks the patient related questions, such as “How would you rate the content of the ‘relaxation’ segment of the module you just completed?”, “Do you feel that you have learned valuable skills from the ‘managing stress’ segment of the module you just completed?”, “Was there anything you didn't like about the ‘improving sleep’ segment of the module you just completed?” A patient may also provide this type of information without solicitation. These types of questions provide one way to quantify the effectiveness of a protocol or a combination of protocols. Indirect response data may also be collected. Continuing the above example, at some point after completion of the above-described module, the patient may be asked questions such as “Do you feel that your stress levels have increased, decreased, or stayed the same since your last session?”, “Has your quality of sleep increased, decreased, or stayed the same since you last session?” These types of questions aren't specifically asking for the patient's direct feedback on a protocol or set of protocols utilized in the therapy, but are instead designed to determine whether the protocols utilized by the therapy have generated the intended result in the patient (e.g. decreased stress and increased quality of sleep would be an indication that the protocols were effective). Additionally, data received from devices such as heart rate monitors, blood pressure monitors, and sleep trackers, may also provide indications as to whether the patient's stress levels have increased or decreased, or whether the patient's quality of sleep has increased or decreased.

In practice, patient feedback, such as patient protocol response data 126, is typically collected in a structured manner using established clinical procedures to ensure the validity of the data interpretation. In various embodiments, the results of the data interpretation may sometimes be referred to as “clinically validated outcome measures,” which may typically be defined as tools that are used in clinical settings to assess the current status of a patient. With respect to the embodiments disclosed herein, analysis of clinically validated outcome measures for a patient can help to determine protocol effectiveness. In various embodiments, an effectiveness rating may be a measure of any number of factors, such as, but not limited to, whether a protocol or a combination of protocols reduces symptom severity, eliminates symptoms, results in improved mood, and/or results in better quality of life for the patient.

In one embodiment, once protocol effectiveness determination module 128 has assigned effectiveness ratings to one or more protocols or combination of protocols, this data may also be stored in a data structure, such as patient database 124, as patient protocol effectiveness data 130, for further use. In one embodiment, the patient protocol effectiveness data 130 generated by protocol effectiveness determination module 128 may also be incorporated into historical protocol effectiveness data 108 of therapeutic protocol database 102.

It should be noted again that the above examples are given for illustrative purposes, and are not intended to limit the scope of the invention as disclosed and claimed herein. One of ordinary skill in the art will readily appreciate that there are many different ways to determine and measure the effectiveness of various protocols and combinations of protocols that are used in administration of a therapy. Application of the historical therapeutic protocols discussed herein typically results in outcome measures that can be clinically validated and thus can reliably be associated with one or more related measures of effectiveness.

As to be expected however, although one particular protocol or combination of protocols may be effective for the general patient population, or for the average patient, the same protocol may not be effective at all for a particular patient, or a particular type of patient. As one simplified example, protocol effectiveness determination module 128 might determine that a protocol utilizing phrase X in a therapy module is only 75% effective when administered in a therapy to patient A, however a protocol utilizing phrase Y in a therapy module is determined to be 90% effective when administered to the same patient A. If the same protocol utilizing phrase X is administered to patient B, it might be found that phrase X is 95% effective for patient B, whereas phrase Y may only be 50% effective for patient B. Thus, it should be clear that the effectiveness ratings of various protocols are likely to vary significantly depending on the characteristics of the particular patient.

It follows then, that in order to train one or more therapeutic protocol effectiveness prediction models, such as therapeutic protocol effectiveness prediction models 160, model training data that accounts for differences in protocol effectiveness among different types of patients must be gathered and assembled. In various embodiments, the system and method disclosed herein utilizes patient profile generation module 134 to build a plurality of patient profiles based on data such as patient data 132 of patient database 124. As used herein, the term “patient data” may include data associated with the patient, for example, patient characteristics such as, but not limited to age, sex, ethnicity, religion, marital status, income level, geographic location, personal and family medical history, including the current medical issue that the therapy is designed to treat.

In one embodiment, patient profile generation module 134 may correlate patient protocol effectiveness data 130 with specific profiles in the plurality of generated patient profiles, and, in one embodiment, may store the patient profile data in a data structure, such as patient profile database 136, for later use. Patient profile generation module 134 may generate any number of patient profiles, which may be characterized by the various combinations of patient characteristics represented by patient data 132. As shown in the illustrative embodiment of FIG. 1A, patient profile data 137 of patient profile database 136 includes type 1 patient profile 138, and type 2 patient profile 144 through type n patient profile 150. In one embodiment, type 1 patient profile 138 includes type 1 patient data 140 and type 1 patient protocol effectiveness data 142, type 2 patient profile 144 includes type 2 patient data 146 and type 2 patient protocol effectiveness data 148, and type n patient profile 150 includes type n patient data 152 and type n patient protocol effectiveness data 154, where n can represent any number of patient profiles, depending on the number of patient groupings that a user of the method and system disclosed herein wishes to create.

As specific illustrative examples, type 1 patient data 140 of type 1 patient profile 138 may describe a patient who is a male, between the ages of 10 and 15, living on the west coast of the United States, who has been diagnosed with irritable bowel syndrome (MS). Type 2 patient data 146 of type 2 patient profile 144 may describe a patient who is a female, between the ages of 55 and 60, living in London, who has been diagnosed with breast cancer. Type n patient data 152 of type n patient profile 150 may describe a patient who is a male, between the ages of 65 and 70, living in the southeastern part of the United States, who has been diagnosed with post-traumatic stress disorder (PTSD). In some embodiments, a patient profile of patient profile data 137 may describe a specific patient instead of a group of patients.

In various embodiments, the patient protocol effectiveness data, such as type 1 patient protocol effectiveness data 142, would represent a measure of how effective particular protocols are for patients of that particular type. In one embodiment, patient protocol effectiveness data may include a list of hundreds, thousands, or millions of protocols and combinations of protocols, each with corresponding data indicating an effectiveness rating for each protocol or combination of protocols. In some embodiments, an effectiveness rating for a protocol among a particular type of patient may be a single number representing an average of the effectiveness ratings for that protocol across all members of the group of patients defined by the patient profile type. In some embodiments an effectiveness rating for a protocol among a particular type of patient may be a range of numbers representing the effectiveness ratings for that protocol across all members of the group of patients defined by the patient profile type. In various other embodiments, a weighting system might be utilized, for instance to give higher weight to effectiveness ratings that are more common than others. For example, a particular protocol might have a wide range of effectiveness values for a particular patient profile type, for example, from 30% to 70% effectiveness, however it may be the case that only one or two patients were associated with the 30% effectiveness rating and only one or two patients were associated with the 70% effectiveness rating, however most patients for that profile type were associated with a 60% rating, and so the 60% rating would receive a higher weight that than the other ratings. It should be noted again here that the above examples are given for illustrative purposes only and are not intended to limit the scope of the invention as disclosed and claimed herein.

In various embodiments, once a plurality of patient profiles are generated and associated with protocol effectiveness data, data correlation module 156 of machine learning training module 155 collects patient profile data 137 from patient profile database 136, and historical therapeutic protocol data 104 from therapeutic protocol database 102 and correlates the data to prepare it for transformation into therapeutic protocol effectiveness model training data 158.

In various embodiments, and largely depending on the machine learning based models used, the patient profile data 137 and/or the historical therapeutic protocol data 104 is processed using various methods known in the machine learning arts to identify elements and to vectorize the patient profile data 137 and/or the historical therapeutic protocol data 104. As a specific illustrative example, in a case where the machine leaning based model is a supervised model, the historical therapeutic protocol data 104 and the patient profile data 137 can be analyzed and processed to identify individual elements found to be indicative of protocol effectiveness among certain types of patients, or among a generalized population of patients. These individual elements are then used to create protocol effectiveness data vectors in multidimensional space, resulting in therapeutic protocol effectiveness model training data 158. Therapeutic protocol effectiveness model training data 158 is then used as input data for training one or more machine learning models, such as therapeutic protocol effectiveness prediction models 160. The protocol effectiveness data for a patient profile that correlates with the protocol effectiveness data vector associated with that patient profile is then used as a label for the resulting vector. In various embodiments, this process is repeated for each protocol defined by historical protocol definition data 106 of therapeutic protocol database 102, and for each patient profile type represented by patient profile data 137 of patient profile database 136. The result is that multiple, often millions, of correlated pairs of protocol effectiveness data vectors and patient profiles (as represented by therapeutic protocol effectiveness model training data 158) are used to train one or more machine learning based models, such as therapeutic protocol effectiveness prediction models 160. Consequently, this process results in the creation of one or more trained therapeutic protocol effectiveness prediction models 162.

Those of skill in the art will readily recognize that there are many different types of machine learning based models known in the art, and as such, it should be noted that the specific illustrative example of a supervised machine learning based model discussed above should not be construed as limiting the embodiments set forth herein.

For instance, in various embodiments, the one or more machine learning based models can be one or more of: supervised machine learning-based models; semi supervised machine learning-based models; unsupervised machine learning-based models; classification machine learning-based models; logistical regression machine learning-based models; neural network machine learning-based models; deep learning machine learning-based models; and/or any other machine learning based models discussed herein, known at the time of filing, or as developed/made available after the time of filing.

As will be discussed in further detail below, in various embodiments, once the therapeutic protocol effectiveness prediction models 160 are trained, they can be used in a variety of ways. In one embodiment, the trained therapeutic protocol effectiveness prediction models 162 can be used to dynamically generate an improved or maximally effective therapeutic protocol for a specific patient, or for a specific type of patient. In one embodiment, in the case of dynamically generating an improved or maximally effective therapeutic protocol for a specific patient, a psychological therapy is selected for administration to the patient. Patient data associated with the patient and patient profile data associated with predefined patient profiles are analyzed to select a patient profile that is the best match for the specific patient, and the selected patient profile data is provided to the trained therapeutic protocol effectiveness prediction models 162. In one embodiment, new therapeutic protocol test data is generated or otherwise obtained, and is also provided to the trained therapeutic protocol effectiveness prediction models 162.

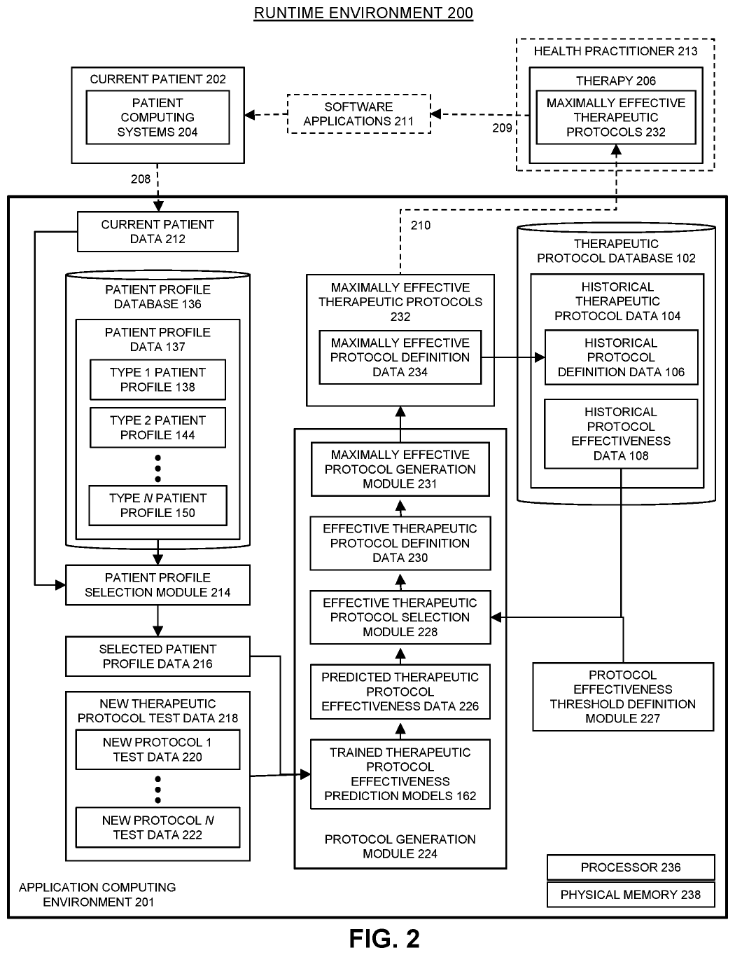

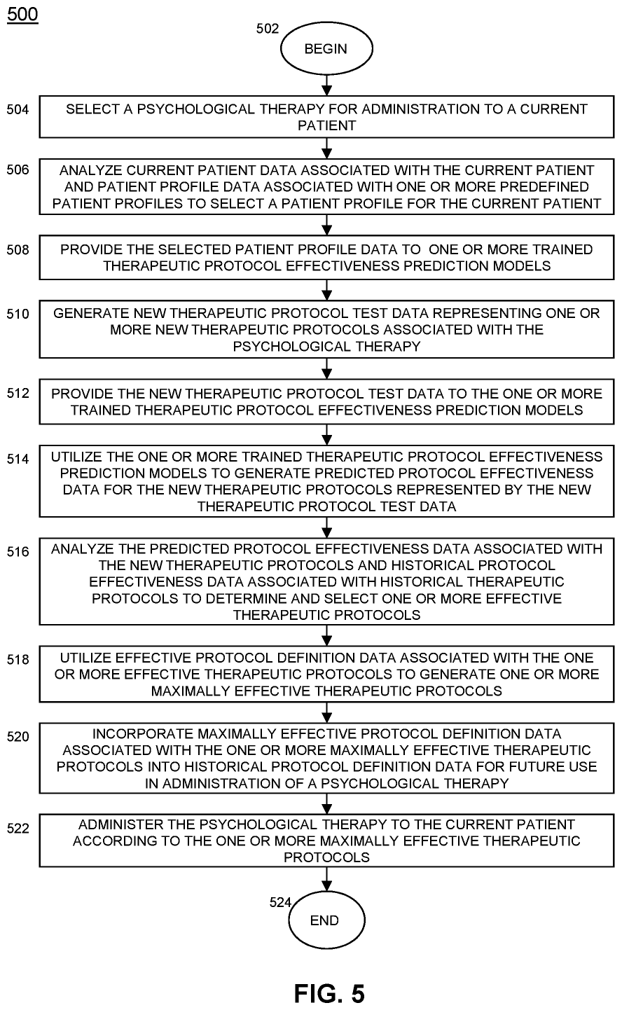

In one embodiment, the trained therapeutic protocol effectiveness prediction models 162 are utilized to generate predicted protocol effectiveness data for the new protocols. In one embodiment, predicted protocol effectiveness data, and historical protocol effectiveness data are analyzed to determine and select one or more effective therapeutic protocols, which are utilized to generate one or more maximally effective therapeutic protocols. In one embodiment, maximally effective protocol definition data associated with the one or more maximally effective therapeutic protocols is stored as historical protocol definition data for future use in administration of a psychological therapy. In one embodiment, the selected psychological therapy is then administered to the patient according to the maximally effective therapeutic protocols. The above described system and process will be discussed in additional detail below with reference to the system of FIG. 2 and the process of FIG. 5.

FIG. 2 is a block diagram of a runtime environment 200 for utilizing trained therapeutic protocol effectiveness prediction models to generate maximally effective therapeutic protocols for a specific patient, in accordance with one embodiment.

In various embodiments, runtime environment 200 includes application computing environment 201, current patient 202 and associated patient computing systems 204, software applications 211, health practitioner 213, therapy 206 and associated maximally effective therapeutic protocols 232. In one embodiment, maximally effective therapeutic protocols 232 include maximally effective protocol definition data 234. In one embodiment, runtime environment 200 further includes communications channel 208, which facilitates transmission of data from current patient 202 to application computing environment 201, communications channel 209, which facilitates administration of therapy 206 to current patient 202, and communications channel 210, which facilitates retrieval of data from application computing environment 201 to be incorporated in therapy 206. Each of the above listed elements will be discussed in further detail below.

In various embodiments, application computing environment 201 includes therapeutic protocol database 102, and patient profile database 136. In one embodiment, therapeutic protocol database 102 includes historical therapeutic protocol data 104, which further includes historical protocol definition data 106, and historical protocol effectiveness data 108. In one embodiment, patient profile database 136 includes patient profile data 137, which further includes type 1 patient profile 138, and type 2 patient profile 144 through type n patient profile 150. Each of the above listed elements will be discussed in further detail below.

In various embodiments, application computing environment 201 further includes additional data such as current patient data 212, selected patient profile data 216, and new therapeutic protocol test data 218, which further includes new protocol 1 test data 220 through new protocol n test data 222. In one embodiment, application computing environment 201 further includes several process modules, such as patient profile selection module 214, protocol generation module 224, and protocol effectiveness threshold definition module 227. In various embodiments, protocol generation module 224 includes trained therapeutic protocol effectiveness prediction models 162, predicted therapeutic protocol effectiveness data 226, effective therapeutic protocol selection module 228, effective therapeutic protocol definition data 230, and maximally effective protocol generation module 231. In one embodiment, application computing environment 201 further includes processor 236 and physical memory 238, which together coordinate the operation and interaction of the data and data processing modules associated with application computing environment 201. Each of the above listed elements will be discussed in further detail below.

In one embodiment, current patient 202 is a patient who has been diagnosed with a medical condition, and a determination is made as to whether current patient 202 will benefit from receiving one or more therapies. As noted above, there are a variety of established and/or clinically validated therapies that have been shown to provide benefit to patients, and administration of these clinically validated therapies are typically governed by a collection of therapeutic protocols associated with the particular therapy. In one embodiment, once a determination has been made that current patient 202 is likely to benefit from a particular therapy, such as therapy 206, the previously trained therapeutic protocol effectiveness prediction models 162 are utilized in order to generate one or more protocols that will be maximally effective for current patient 202.

In order to achieve this outcome, in one embodiment, current patient data 212 is obtained, either directly from current patient 202 and/or patient computing systems 204, from medical files associated with current patient 202 and/or current patient data 212 may be retrieved from a database of previously collected patient data. As noted above, and as used herein, the term “patient data” may include data associated with the patient, for example, patient characteristics such as, but not limited to age, sex, ethnicity, religion, marital status, income level, geographic location, personal and family medical history, including the current medical issue that the therapy is designed to treat. In various embodiments, patient computing systems 204 may include, but are not limited to, a desktop computing system, a mobile computing system, a virtual reality computing system, a gaming computing system, a computing system that utilizes one or more Internet of Things (IoT) devices, or any combination thereof.

In some embodiments, there may be no specific current patient 202, and test data may be used in place of current patient data 212. For example, a theoretical patient may be contemplated, and data describing characteristics of the theoretical patient may be used as test data in place of current patient data 212 to generate maximally effective therapeutic protocols for the theoretical patient. In various embodiments, test data may be generated by one or more machine learning models that have been trained to predict effectiveness of the test data.

In various embodiments, once the current patient data 212 has been obtained for current patient 202, patient profile selection module 214 analyzes the current patient data 212 along with the patient profile data 137 of patient profile database 136, in order to select a patient profile that most closely matches the characteristics of current patient 202.

In one embodiment, patient profile selection module 214 compares various characteristics of current patient 202 to patient characteristics represented by the one or more patient profiles in the patient profile database 136. As will be noted by those of skill in the art, various mechanisms and algorithms may be utilized to determine similarities between current patient 202 and the patient profiles represented by patient profile data 137. Similarity of current patient 202 to a particular patient profile may be determined by any number of factors, such as, but not limited to current patient 202's age, sex, ethnicity, religion, marital status, income level, geographic location, personal and family medical history, including the current medical issue that the therapy is designed to treat.

In one embodiment, one or more thresholds may be defined to determine how close of a match current patient 202 is to a particular patient profile. For example, if current patient 202's characteristics match with type 1 patient profile 138 by 60%, but match with type 2 patient profile 144 by 80%, and no other patient profile type is found to provide a better match, the patient profile with the closest match may be selected for utilization in determination of an effective therapeutic protocol to utilize for treating current patient 202.

Continuing the specific illustrative examples given above, current patient 202 may be a male, age 13, living in California, who has been diagnosed with IBS, and so may be classified as a ‘type 1’ patient, wherein the ‘type 1’ patient is associated with type 1 patient profile 138, which describes a patient who is a male, between the ages of 10 and 15, living on the west coast of the United States, who has been diagnosed with IBS, and so patient profile selection module 214 may determine that current patient 202 should be associated with type 1 patient profile 138, and this selection may be represented by selected patient profile data 216. It should be noted herein that the above examples are simplified, and are given for illustrative purposes only. One of skill in the art will readily recognize that the millions of different combinations of patient characteristics, the models that govern the interactions between those characteristics, and the protocols associated with the treatment administered to those patients, requires a vast amount of data collection and analysis which simply cannot be performed by the human mind alone, even with the aid of pen and paper and even given unlimited time to accomplish the task.

In various embodiments, once patient profile selection module 214 has determined selected patient profile data 216, the selected patient profile data 216 is provided as input to the one or more trained therapeutic protocol effectiveness prediction models 162, along with new therapeutic protocol test data 218. As discussed above, in various embodiments, and as used herein, the terms “current therapeutic protocol” or “historical therapeutic protocol” refer to a protocol that has previously been generated, tested, established, and/or clinically validated for use in administration of a therapy. Likewise, in one embodiment, the terms “new protocol,” and “new therapeutic protocol” refer to a protocol that has not been previously generated, tested, established, and/or clinically validated for use in administration of a therapy. Additionally, in some embodiments, the terms “new protocol,” and “new therapeutic protocol” may refer to a protocol that has been previously generated and/or tested, but may not yet be established and/or clinically validated for use in administration of a therapy. In various embodiments, new therapeutic protocols may also be thought of as potential therapeutic protocols or candidate therapeutic protocols, in the sense that they are protocols that are being considered for use in a therapy.

As one simplified example, a historical therapeutic protocol for a cognitive behavioral therapy may dictate that the therapy should contain eight modules that are presented to the patient in a particular order, such as 1,2,3,4,5,6,7,8. A new therapeutic protocol might dictate that there should be nine modules, presented to the patient in a different order, such as 1,2,4,3,6,5,8,7,9. Similarly, a historical therapeutic protocol for a cognitive behavioral therapy may dictate that a page section of module 6 should include text content that contains the word sequence “alternatives to negative thoughts,” whereas a new therapeutic protocol may dictate that the same page section of module 6 should instead include text content that contains the word sequence “alternatives to unhelpful thoughts.”

In one embodiment, new therapeutic protocol test data 218 of FIG. 2A includes data representing any number of new therapeutic protocols, such as new protocol 1 through new protocol n, which are represented by new protocol 1 test data 220 through new protocol n test data 222. In one embodiment, once the selected patient profile data 216 and new therapeutic protocol test data 218 have been provided as input to the one or more trained therapeutic protocol effectiveness prediction models 162 of protocol generation module 224, the one or more trained therapeutic protocol effectiveness prediction models 162 generate predicted therapeutic protocol effectiveness data 226. In various embodiments, predicted therapeutic protocol effectiveness data 226 represents the predicted effectiveness of each of the new therapeutic protocols represented by new therapeutic protocol test data 218 for a patient who matches the patient profile type represented by selected patient profile data 216.

As one simplified example, patient profile selection module 214 might categorize current patient 202 as a match for a type 1 patient, as represented by type 1 patient profile 138 of patient profile data 137. Predicted therapeutic protocol effectiveness data 226 might indicate that a first new protocol is 75% effective for type 1 patients, and a second new protocol is 50% effective for type 1 patients. Likewise, for a patient who has been categorized as a type 2 patient, predicted therapeutic protocol effectiveness data 226 might indicate that the same first new protocol is 30% effective for type 2 patients, and the same second new protocol is 90% effective for type 2 patients. Thus, the output of the trained therapeutic protocol effectiveness prediction models 162, predicted therapeutic protocol effectiveness data 226, is dependent on both the new therapeutic protocol test data 218, as well as the patient profile type represented by selected patient profile data 216.

As discussed above, effectiveness of a protocol or a combination of protocols can be determined and defined in a number of ways, including, but not limited to, analysis of direct or indirect feedback from a patient, analysis of patient physiological data, and/or analysis of a variety of clinically validated outcome measures. An effectiveness rating may be a measure of any number of factors, such as, but not limited to, whether a protocol or a combination of protocols reduces symptom severity, eliminates symptoms, results in improved mood, and/or results in better quality of life for the patient.

In one embodiment, once predicted therapeutic protocol effectiveness data 226 has been generated by trained therapeutic protocol effectiveness prediction models 162, it is passed to effective therapeutic protocol selection module 228 of protocol generation module 224 for further analysis. In one embodiment, effective therapeutic protocol selection module 228 selects one or more of the new therapeutic protocols represented by new therapeutic protocol test data 218 that have been found to be effective. A determination as to what constitutes an “effective” protocol may be made in any number of ways. As one illustrative example, protocol effectiveness threshold definition module 227 may set one or more threshold values for the effectiveness ratings represented by predicted therapeutic protocol effectiveness data 226. In one embodiment, protocol effectiveness threshold definition module 227 may be separate from protocol generation module 224. In one embodiment, protocol effectiveness threshold definition module 227 may be a sub-module of protocol generation module 224. In one embodiment, one or more threshold values may be explicitly set, for example, based on input from one or more health practitioners. In various other embodiments, protocol effectiveness threshold definition module 227 may derive or learn one or more threshold values based on analysis of training data, such as, but not limited to historical protocol effectiveness data 108. As one simplified example, in one embodiment, protocol effectiveness threshold definition module 227 may define an effectiveness threshold such that any protocol having a known or predicted effectiveness rating of 75% or higher should be considered an “effective” protocol by effective therapeutic protocol selection module 228. In one embodiment, effective therapeutic protocol selection module 228 may also consider historical protocol effectiveness data 108 in determining and selecting effective protocols.

Continuing the above simplified example, a historical therapeutic protocol for a cognitive behavioral therapy may dictate that the therapy should contain eight modules that are presented to the patient in a particular order, such as 1,2,3,4,5,6,7,8. A new therapeutic protocol might dictate that there should be nine modules, presented to the patient in a different order, such as 1,2,4,3,6,5,8,7,9. It may be found that the historical protocol has a known effectiveness rating of 90%, whereas the new therapeutic protocol has a predicted effectiveness rating of 80%. In the illustrative embodiment where protocol effectiveness threshold definition module 227 has set the threshold value for effectiveness ratings to 75%, the effective therapeutic protocol selection module 228 may determine that, while the new protocol is predicted to be effective, the historical protocol is actually known to be more effective, and so the historical protocol may be selected over the new protocol. Similarly, a historical therapeutic protocol for a cognitive behavioral therapy may dictate that a page section of module 6 should include text content that contains the word sequence “alternatives to negative thoughts,” whereas a new therapeutic protocol may dictate that the same page section of module 6 should instead include text content that contains the word sequence “alternatives to unhelpful thoughts.” The historical protocol may have a known 75% effectiveness rating, but the new protocol may have a predicted 85% rating, and so effective therapeutic protocol selection module 228 may select the new protocol.

In one embodiment, once effective therapeutic protocol selection module 228 has selected one or more effective therapeutic protocols, effective therapeutic protocol definition data 230 is generated, which contains data defining the one or more selected effective protocols. In one embodiment, maximally effective protocol generation module 231 utilizes effective therapeutic protocol definition data 230 to generate one or more maximally effective therapeutic protocols 232. As used herein, the term “maximally effective protocol” or “maximally effective therapeutic protocol” may include therapeutic protocols that have been determined to be the most effective therapeutic protocols, for a particular period of time, out of the new, current, and/or historical effective therapeutic protocols. In various embodiments, maximally effective therapeutic protocols 232 may include any number and combination of maximally effective protocols, and each of these protocols or protocol combinations is defined by maximally effective protocol definition data 234. Continuing the above illustrative example, protocol generation module 224 may determine that using the word sequence “alternative to unhelpful thoughts” in place of “alternative to negative thoughts” is maximally effective for the patient profile type represented by selected patient profile data 216, independently of the other protocols in the therapy. Protocol generation module 224 may instead determine that using the word sequence “alternative to unhelpful thoughts” in place of “alternative to negative thoughts” is only effective when presented on page one of module six of a therapy that has eight modules.

It should be noted here that the above simplified examples are given for illustrative purposes only and are not intended to limit the invention as disclosed and claimed herein. It should be readily apparent to those of ordinary skill in the art that there are millions of potential protocols and protocol combinations that may be employed in a therapy, and so the generation of maximally effective protocols and protocol combinations is not a task that can be accomplished in the human mind, even with pen and paper, and even given unlimited time.

Referring briefly to FIG. 1A and FIG. 2 together, in various embodiments, once one or more maximally effective therapeutic protocols 232 have been generated by maximally effective protocol generation module 231 of protocol generation module 224, the maximally effective protocol definition data 234 representing the maximally effective therapeutic protocols 232 may be stored in a data structure, such as therapeutic protocol database 102, for further use. For example, in one embodiment, maximally effective protocol definition data 234 is incorporated into historical protocol definition data 106 of historical therapeutic protocol data 104. This is advantageous because it creates a feedback loop for the machine learning process, wherein the newly generated maximally effective therapeutic protocols 232 can be incorporated into the therapeutic protocol effectiveness model training data 158, of FIG. 1A, which is used to train the therapeutic protocol effectiveness prediction models 160. In this manner, the trained therapeutic protocol effectiveness prediction models 162 may be continually updated and refined as new patient protocol response data 126 is received from patient 118.

In one embodiment, once one or more maximally effective therapeutic protocols 232 have been generated by maximally effective protocol generation module 231 of protocol generation module 224, the one or more maximally effective therapeutic protocols 232 may be incorporated into a therapy, such as therapy 206, which may then be administered to a patient, such as current patient 202. In some embodiments, once generated, the maximally effective therapeutic protocols 232 may be automatically incorporated into a therapy, such as therapy 206, for administration to current patient 202. In some embodiments, a health practitioner, such as health practitioner 213, may review maximally effective therapeutic protocols 232 prior to incorporation into therapy 206 for administration to current patient 202. In some embodiments, the maximally effective therapeutic protocols 232 may be stored in a data structure, such as therapeutic protocol database 102, for further use, but might not be incorporated into a particular therapy. In some embodiments, the one or more maximally effective therapeutic protocols 232 may be incorporated into a therapy, such as therapy 206, but the therapy 206 may not be administered to current patient 202 and/or the therapy 206 may be administered to a patient other than current patient 202.

In various embodiments, the therapy 206 may be administered to current patient 202 using one or more communication mechanisms 209. In some embodiments, communication mechanisms 209 include health practitioner 213 conducting a physical in-person meeting with current patient 202 to verbally guide current patient 202 through the therapy 206. In other embodiments, communication mechanisms 209 include administering the therapy 206 to current patient 202 remotely, for example through a website, or through one or more software applications 211 that can be executed from patient computing systems 204. In one embodiment, the therapy 206 may be administered to current patient 202 directly by health practitioner 213. In one embodiment, therapy 206 may be administered to current patient 202 remotely, without the direct involvement of health practitioner 213. For example, therapy 206 may be self-administered by current patient 202. In one embodiment, therapy 206 may also be administered to current patient 202 remotely with partial involvement of health practitioner 213.

In various embodiments, patient computing systems 204 may include, but are not limited to, a desktop computing system, a mobile computing system, a virtual reality computing system, a gaming computing system, a computing system that utilizes one or more Internet of Things (IoT) devices, or any combination thereof.

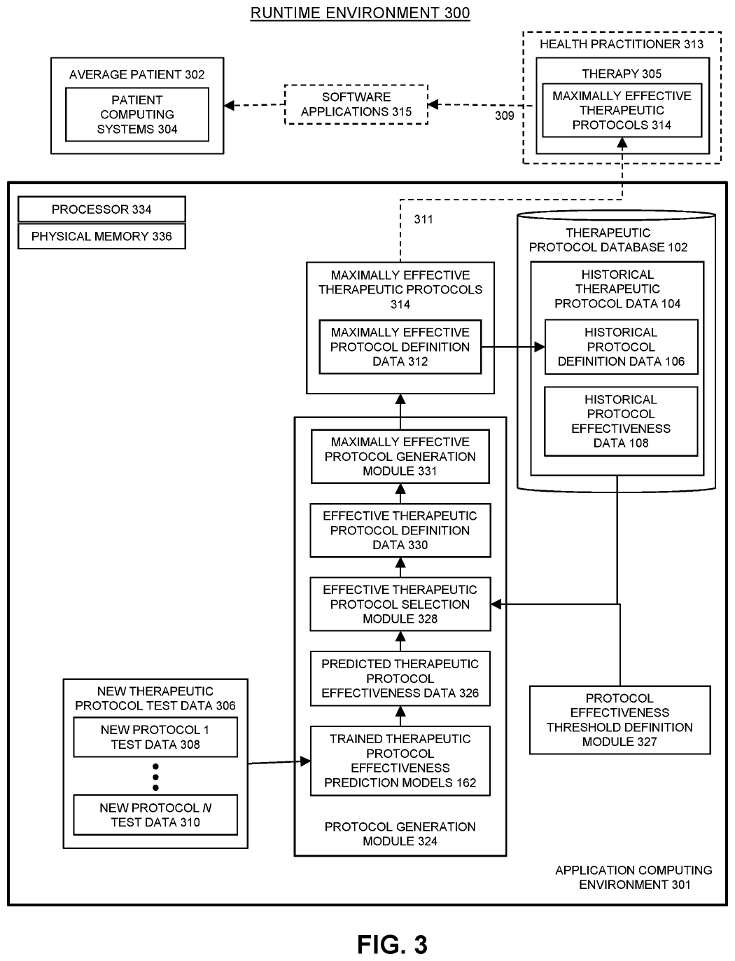

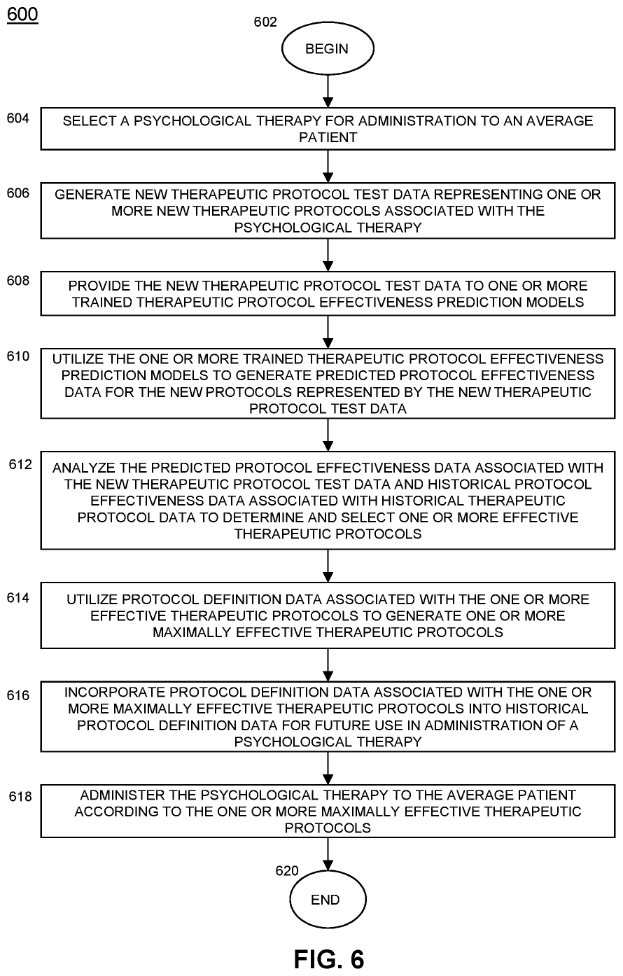

As will be discussed in further detail below, in addition to the embodiments discussed above, trained therapeutic protocol effectiveness prediction models 162 can be utilized independently of a specific patient or specific type of patient, for example, to generate one or more improved or maximally effective therapeutic protocols that are generally effective for patients, regardless of the patients' background and history. In one embodiment, in the case of generating improved or maximally effective therapeutic protocols that are generally effective for patients, or are effective for an average patient, a system similar to that described above may be utilized, without providing patient-specific profile data to the trained therapeutic protocol effectiveness prediction models. For example, in some embodiments, a psychological therapy is selected for administration to one or more patients, new therapeutic protocol test data is generated and provided to the trained therapeutic protocol effectiveness prediction models 162, and the trained therapeutic protocol effectiveness prediction models 162 are utilized to generate predicted protocol effectiveness data for the new protocols. In some embodiments, the predicted protocol effectiveness data and the historical protocol effectiveness data are analyzed to select one or more effective therapeutic protocols. Protocol definition data associated with the one or more effective therapeutic protocols is then utilized to generate one or more maximally effective therapeutic protocols. In one embodiment, maximally effective protocol definition data associated with the one or more maximally effective therapeutic protocols is stored as historical protocol definition data for future use in administration of the selected psychological therapy. The above described system and process will be discussed in additional detail below with reference to the system of FIG. 3 and the process of FIG. 6.

FIG. 3 is a block diagram of a runtime environment 300 for utilizing trained therapeutic protocol effectiveness prediction models to generate generalized maximally effective therapeutic protocols, in accordance with one embodiment.

In various embodiments, runtime environment 300 includes application computing environment 301, average patient 302 and associated patient computing systems 304, software applications 315, health practitioner 313, therapy 305, and maximally effective therapeutic protocols 314. In one embodiment, runtime environment 300 further includes communications channel 309, which facilitates administration of therapy 305 to average patient 302, and communications channel 311, which facilitates retrieval of data from application computing environment 301. In one embodiment, application computing environment 301 includes therapeutic protocol database 102, which further includes historical therapeutic protocol data 104, such as historical protocol definition data 106, and historical protocol effectiveness data 108. In various embodiments, application computing environment 301 further includes additional data such as new therapeutic protocol test data 306, which further includes new protocol 1 test data 308 through new protocol n test data 310. In one embodiment, application computing environment 301 further includes protocol generation module 324, and protocol effectiveness threshold definition module 327. In various embodiments, protocol generation module 324 includes trained therapeutic protocol effectiveness prediction models 162, predicted therapeutic protocol effectiveness data 326, effective therapeutic protocol selection module 328, effective therapeutic protocol definition data 330, and maximally effective protocol generation module 331. In one embodiment, application computing environment 301 further includes processor 334 and physical memory 336, which together coordinate the operation and interaction of the data and data processing modules associated with application computing environment 301. Each of the above listed elements will be discussed in further detail below.

As noted above, there are a variety of established and/or clinically validated therapies that have been shown to provide benefit to patients, and administration of these clinically validated therapies are typically governed by a collection of therapeutic protocols associated with the particular therapy. In one embodiment, a therapy, such as therapy 305, is selected for administration to one or more patients, and the previously trained therapeutic protocol effectiveness prediction models 162 are utilized in order to generate one or more protocols that will be maximally effective for average patients or for patients in general.

In one embodiment, new therapeutic protocol test data 306 is generated or otherwise obtained, and is provided as input to the one or more trained therapeutic protocol effectiveness prediction models 162 of protocol generation module 324. In one embodiment, new therapeutic protocol test data 306 of FIG. 3 includes data representing any number of new therapeutic protocols, such as new protocol 1 through new protocol n, which are represented by new protocol 1 test data 308 through new protocol n test data 310. In one embodiment, once the new therapeutic protocol test data 306 has been provided as input to the one or more trained therapeutic protocol effectiveness prediction models 162 of protocol generation module 324, the one or more trained therapeutic protocol effectiveness prediction models 162 generate predicted therapeutic protocol effectiveness data 326. In various embodiments, predicted therapeutic protocol effectiveness data 326 represents the predicted effectiveness of each of the new therapeutic protocols represented by new therapeutic protocol test data 306.

In one embodiment, once predicted therapeutic protocol effectiveness data 326 has been generated by trained therapeutic protocol effectiveness prediction models 162, it is passed to effective therapeutic protocol selection module 328 of protocol generation module 324 for further analysis. In one embodiment, effective therapeutic protocol selection module 328 selects one or more of the new therapeutic protocols represented by new therapeutic protocol test data 306 that have been found to be effective. As discussed above, a determination as to what constitutes an “effective” protocol may be made in any number of ways. As one illustrative example, protocol effectiveness threshold definition module 327 may set one or more threshold values for the effectiveness ratings represented by predicted therapeutic protocol effectiveness data 326. In one embodiment, protocol effectiveness threshold definition module 327 may be separate from protocol generation module 324. In one embodiment, protocol effectiveness threshold definition module 327 may be a sub-module of protocol generation module 324. In one embodiment, one or more threshold values may be explicitly set, for example, based on input from one or more health practitioners. In various other embodiments, protocol effectiveness threshold definition module 327 may derive or learn one or more threshold values based on analysis of training data, including, but not limited to historical protocol effectiveness data 108. In one embodiment, effective therapeutic protocol selection module 328 may also consider historical protocol effectiveness data 108 in determining and selecting effective protocols.

In one embodiment, once effective therapeutic protocol selection module 328 has selected one or more effective therapeutic protocols, effective therapeutic protocol definition data 330 is generated, which contains data defining the one or more selected effective protocols. In one embodiment, maximally effective protocol generation module 331 utilizes effective therapeutic protocol definition data 330 to generate one or more maximally effective therapeutic protocols 314. In various embodiments, maximally effective therapeutic protocols 314 may include any number and combination of maximally effective protocols, and each of these protocols or protocol combinations is defined by maximally effective protocol definition data 312.

Referring briefly to FIG. 1A and FIG. 3 together, in various embodiments, once one or more maximally effective therapeutic protocols 314 have been generated by maximally effective protocol generation module 331 of protocol generation module 324, the maximally effective protocol definition data 312 representing the maximally effective therapeutic protocols 314 may be stored in a data structure, such as therapeutic protocol database 102, for further use. For example, in one embodiment, maximally effective protocol definition data 312 is stored as historical protocol definition data 106 of historical therapeutic protocol data 104. As noted above, this is advantageous because it creates a feedback loop for the machine learning process, wherein the newly generated maximally effective therapeutic protocols 314 can be incorporated into the therapeutic protocol effectiveness model training data 158 of FIG. 1A, which is used to train the therapeutic protocol effectiveness prediction models 160. In this manner, the trained therapeutic protocol effectiveness prediction models 162 may be continually updated and refined as new patient protocol response data 126 is received from patient 118.

In one embodiment, once one or more maximally effective therapeutic protocols 314 have been generated by maximally effective protocol generation module 331 of protocol generation module 324, the one or more maximally effective therapeutic protocols 314 may be incorporated into a therapy, such as therapy 305. In some embodiments, the maximally effective therapeutic protocols 232 may be stored in a data structure such as therapeutic protocol database 102, for further use, but might not be incorporated into a particular therapy.

In one embodiment, once maximally effective therapeutic protocols 314 have been incorporated into a therapy, such as therapy 305, therapy 305 may then be administered to a patient, such as average patient 302. In some embodiments, once generated, the maximally effective therapeutic protocols may be automatically incorporated into therapy 305 for administration to average patient 302. In some embodiments, a health practitioner, such as health practitioner 313, may review maximally effective therapeutic protocols 314 prior to incorporation into therapy 305 for administration to average patient 302. In some embodiments, the maximally effective therapeutic protocols 314 may be stored in a data structure, such as therapeutic protocol database 102, for further use, but might not be administered to average patient 302.

In various embodiments, the maximally effective therapeutic protocols 314 may be administered to average patient 302 using one or more communication mechanisms 309. In some embodiments, communication mechanisms 309 include health practitioner 313 conducting a physical in-person meeting with average patient 302 to verbally guide average patient 302 through the therapy 305. In other embodiments, communication mechanisms 309 include administering the therapy 305 to average patient 302 remotely, for example through a website, or through one or more software applications 315 that can be executed from patient computing systems 304. In one embodiment, the therapy 305 may be administered to average patient 302 directly by health practitioner 313. In one embodiment, therapy 305 may be administered to average patient 302 remotely, without the direct involvement of health practitioner 313. For example, therapy 305 may be self-administered by average patient 302. In one embodiment, therapy 305 may also be administered to average patient 302 remotely with partial involvement of health practitioner 313.

In various embodiments, patient computing systems 304 may include, but are not limited to, a desktop computing system, a mobile computing system, a virtual reality computing system, a gaming computing system, a computing system that utilizes one or more Internet of Things (IoT) devices, or any combination thereof.

### Process

FIG. 4 is a flow chart of a process 400 for creating trained therapeutic protocol effectiveness prediction models, in accordance with one embodiment.

Process 400 begins at BEGIN 402 and process flow proceeds to 404. At 404, a psychological therapy is selected for administration to one or more patients.

In one embodiment, the one or more patients are patients who have been diagnosed with a medical condition, and a determination is made as to whether the one or more patients will benefit from receiving one or more therapies. In one embodiment, once a determination has been made that one or more patients are likely to benefit from a particular therapy the therapy is selected for administration to the one or more patients.

In one embodiment, once a psychological therapy is selected for administration to one or more patients at 404, process flow proceeds to 406. In one embodiment, at 406, the selected psychological therapy is administered to the one or more patients according to one or more historical therapeutic protocols associated with the selected psychological therapy.

As noted above, there are a variety of established and/or clinically validated therapies that have been shown to provide benefit to patients, and administration of these clinically validated therapies are typically governed by a collection of therapeutic protocols associated with the particular therapy. For any given therapy, associated protocols can be defined and applied to the therapy as a whole, or to any individual component or sub-component of a therapy. In various embodiments, a determination may be made as to which historical therapeutic protocols to use for administration of the therapy to the one or more patients. This determination is at the discretion of the health care practitioner responsible for each patient's treatment, and the determination may be based on a wide variety of factors, such as, but not limited to, the severity of the patient's symptoms, the patient's medical history, the patient's age group, the patient's sex, and the patient's ethnicity.

In one embodiment, once the selected psychological therapy is administered to the one or more patients at 406, process flow proceeds to 408. In one embodiment, at 408, the patient responses to the historical therapeutic protocols are monitored to obtain patient protocol response data.

In various embodiments, patient protocol response data may include direct verbal or written feedback, and/or indirect feedback, such as an indication of whether a particular therapeutic protocol appears to be having an effect on the patient. The patient protocol response data may further include any other measureable data such as, but not limited to, click-stream data showing details related to patient engagement with the content of the therapy, for instance, the time that the patient spends engaging with each section of a particular therapy module. The patient protocol response data may also include data received from devices such as, but not limited to, sleep trackers, or other types of physiological sensors that may be used to measure a patient's physiological state, such as heart rate, respiratory rate, and/or blood pressure.

In one embodiment, once patient protocol response data is obtained at 408, process flow proceeds to 410. In one embodiment, at 410, the patient protocol response data is analyzed to determine the effectiveness of the one or more historical therapeutic protocols for the one or more patients.

In various embodiments, effectiveness of therapeutic protocols for the one or more patients may be defined and determined in a variety of ways based on the patient protocol response data. For example, in practice, patient protocol response data is typically collected in a structured manner using established clinical procedures to ensure the validity of the data interpretation. In various embodiments, the results of the data interpretation may sometimes be referred to as “clinically validated outcome measures,” which may typically be defined as tools that are used in clinical settings to assess the current status of a patient. With respect to the embodiments disclosed herein, analysis of clinically validated outcome measures for a patient can help to determine protocol effectiveness.

In one embodiment, once effectiveness of the one or more historical therapeutic protocols is determined for the one or more patients at 410, process flow proceeds to 412. In one embodiment, at 412, patient protocol effectiveness data is generated representing the effectiveness of the one or more therapeutic protocols for the one or more patients.

As detailed in the system discussion above, in various embodiments, at 412, effectiveness ratings are assigned to one or more protocols or combination of protocols, and the resulting protocol effectiveness data may be stored in one or more data structures for further use.

In one embodiment, once patient protocol effectiveness data is generated at 412, process flow proceeds to 414. In one embodiment, at 414, the patient protocol effectiveness data and patient data associated with the patient are analyzed to generate one or more patient profiles.

As discussed above, although one particular protocol or combination of protocols may be effective for the general patient population, or for the average patient, the same protocol may not be effective at all for a particular patient, or a particular type of patient, and as such, the effectiveness ratings of various protocols are likely to vary significantly depending on the characteristics of the particular patient. It follows then, that in order to train one or more therapeutic protocol effectiveness prediction models, model training data that accounts for differences in protocol effectiveness among different types of patients must be gathered and assembled. In various embodiments, the system and method disclosed herein builds a plurality of patient profiles based on known or obtained patient data. Patient data may include, for example, patient characteristics such as, but not limited to age, sex, ethnicity, religion, marital status, income level, geographic location, personal and family medical history, including the current medical issue that the therapy is designed to treat.

In one embodiment, patient protocol effectiveness data is correlated with specific profiles in the plurality of generated patient profiles, and the patient profile data may be stored in one or more data structures for later use. Any number of patient profiles may be generated, and the patient profiles are typically characterized by a combination of patient characteristics represented by the known or obtained patient data.

In various embodiments, the patient protocol effectiveness data represents a measure of how effective particular protocols are for patients of that particular type. In one embodiment, patient protocol effectiveness data may include a list of hundreds, thousands, or millions of protocols and combinations of protocols, each with corresponding data indicating an effectiveness rating for each protocol or combination of protocols. In some embodiments, an effectiveness rating for a protocol among a particular type of patient may be a single number representing an average of the effectiveness ratings for that protocol across all members of the group of patients defined by the patient profile type. In some embodiments an effectiveness rating for a protocol among a particular type of patient may be a range of numbers representing the effectiveness ratings for that protocol across all members of the group of patients defined by the patient profile type. In various other embodiments, a weighting system might be utilized, for instance to give higher weight to effectiveness ratings that are more common than others.

In one embodiment, once one or more patient profiles are generated at 414, process flow proceeds to 416. In one embodiment, at 416, historical therapeutic protocol data associated with the one or more historical therapeutic protocols is correlated with patient profile data associated with the one or more patient profiles to generate therapeutic protocol effectiveness model training data.

In various embodiments, once a plurality of patient profiles are generated and associated with protocol effectiveness data, patient profile data and historical therapeutic protocol data is collected and the data is correlated to prepare it for transformation into therapeutic protocol effectiveness model training data for training one or more therapeutic protocol effectiveness models.

In one embodiment, once therapeutic protocol effectiveness model training data is generated at 416, process flow proceeds to 418. In one embodiment, at 418, the therapeutic protocol effectiveness model training data is used to train one or more machine learning based therapeutic protocol effectiveness prediction models, thereby resulting in the creation of one or more trained therapeutic protocol effectiveness prediction models.

In various embodiments, and largely depending on the machine learning based models used, the patient profile data and/or the historical therapeutic protocol data is processed using various methods know in the machine learning arts to identify elements and to vectorize the patient profile data and/or the historical therapeutic protocol data. As a specific illustrative example, in a case where the machine leaning based model is a supervised model, the historical therapeutic protocol data and the patient profile data can be analyzed and processed to identify individual elements found to be indicative of protocol effectiveness among certain types of patients, or among a generalized population of patients. These individual elements are then used to create protocol effectiveness data vectors in multidimensional space, resulting in therapeutic protocol effectiveness model training data. The therapeutic protocol effectiveness model training data is then used as input data for training one or more therapeutic protocol effectiveness prediction models. The protocol effectiveness data for a patient profile that correlates with the protocol effectiveness data vector associated with that patient profile is then used as a label for the resulting vector. In various embodiments, this process is repeated for each protocol defined by the historical protocol definition data, and for each patient profile type represented by the patient profile data. The result is that multiple, often millions, of correlated pairs of protocol effectiveness data vectors and patient profiles are used to train the therapeutic protocol effectiveness prediction models. Consequently, this process results in the creation of one or more trained therapeutic protocol effectiveness prediction models.

In one embodiment, once one or more trained therapeutic protocol effectiveness prediction models are created at 418, process flow proceeds to 420. In one embodiment, at 420, a determination is made as to whether the one or more therapeutic protocol effectiveness prediction models should continue to be trained. In various embodiments, this determination may be made at the discretion of an operator or administrator of the system and method disclosed herein.

In one embodiment, upon a determination at 420 that the one or more therapeutic protocol effectiveness prediction models should continue to be trained, process flow returns to 404, and the above described operations may be repeated indefinitely.

In one embodiment, upon a determination at 420 that the one or more therapeutic protocol effectiveness prediction models should not continue to be trained, process flow proceeds to END 422 and the process 400 for creating trained therapeutic protocol effectiveness prediction models is exited to await new data and/or instructions.

FIG. 5 is a flow chart of a process 500 for utilizing trained therapeutic protocol effectiveness prediction models to generate maximally effective therapeutic protocols for a specific patient, in accordance with one embodiment.

Process 500 begins at BEGIN 502 and process flow proceeds to 504. At 504, a psychological therapy is selected for administration to a current patient.

In one embodiment, the current patient is a patient who has been diagnosed with a medical condition, and a determination is made as to whether the current patient will benefit from receiving one or more therapies. As noted above, there are a variety of established and/or clinically validated therapies that have been shown to provide benefit to patients, and administration of these clinically validated therapies are typically governed by a collection of therapeutic protocols associated with the particular therapy. In one embodiment, once a determination has been made that the current patient is likely to benefit from a particular therapy, that therapy is selected for administration to the current patient, and the previously trained therapeutic protocol effectiveness prediction models are utilized in order to generate one or more protocols that will be maximally effective for the current patient.

In one embodiment, once a psychological therapy is selected for administration to the current patient at 504, process flow proceeds to 506. In one embodiment, at 506, current patient data associated with the current patient and patient profile data associated with one or more predefined patient profiles are analyzed to select a patient profile that is the best match for the current patient.

In one embodiment, the current patient data is obtained, either directly from the current patient, from medical files associated with current patient, and/or current patient data may be retrieved from a database of previously collected patient data. In some embodiments, there may be no specific current patient, and test data may be used in place of current patient data. For example, a theoretical patient may be contemplated, and data describing characteristics of the theoretical patient may be used as test data in place of current patient data to generate maximally effective therapeutic protocols for the theoretical patient. In various embodiments, test data may be generated by one or more machine learning models that have been trained to predict effectiveness of the test data.

In various embodiments, once the current patient data has been obtained for the current patient, the current patient data is analyzed along with the patient profile data, in order to select a patient profile that most closely matches the characteristics of the current patient. In one embodiment, various characteristics of the current patient are compared to patient characteristics represented by the one or more patient profiles. As will be noted by those of skill in the art, various mechanisms and algorithms may be utilized to determine similarities between the current patient and the patient profiles represented by patient profile data. Similarity of the current patient to a particular patient profile may be determined by any number of factors, such as, but not limited to the current patient's age, sex, ethnicity, religion, marital status, income level, geographic location, personal and family medical history, including the current medical issue that the therapy is designed to treat. In one embodiment, one or more thresholds may be defined to determine how close of a match the current patient is to a particular patient profile.

In one embodiment, once a patient profile is selected at 506, process flow proceeds to 508. In one embodiment, at 508, the selected patient profile data is provided as input to one or more trained therapeutic protocol effectiveness prediction models.

In one embodiment, once the patient profile data is provided to one or more trained therapeutic protocol effectiveness prediction models at 508, process flow proceeds to 510. In one embodiment, at 510, new therapeutic protocol test data is generated representing one or more new therapeutic protocols associated with the psychological therapy.

In various embodiments, new therapeutic protocol test data is generated representing one or more new therapeutic protocols. As noted above, in various embodiments, new therapeutic protocols may also be thought of as potential therapeutic protocols or candidate therapeutic protocols, in the sense that they are protocols that are being considered for use in a therapy. In one embodiment, the new therapeutic protocol test data includes data representing any number of new therapeutic protocols.

In one embodiment, once new therapeutic protocol test data is generated at 510, process flow proceeds to 512. In one embodiment, at 512, the new therapeutic protocol test data is provided as input to the one or more trained therapeutic protocol effectiveness prediction models.

In one embodiment, once the new therapeutic protocol test data is provided as input to the one or more trained therapeutic protocol effectiveness prediction models at 512, process flow proceeds to 514. In one embodiment, at 514, the one or more trained therapeutic protocol effectiveness prediction models are utilized to generate predicted protocol effectiveness data for the new protocols represented by the new therapeutic protocol test data.

In various embodiments, the predicted therapeutic protocol effectiveness data represents the predicted effectiveness of each of the new therapeutic protocols represented by the new therapeutic protocol test data for a patient who matches the patient profile type represented by the selected patient profile data.

In one embodiment, once predicted protocol effectiveness data is generated at 514, process flow proceeds to 516. In one embodiment, at 516, the predicted protocol effectiveness data associated with the new therapeutic protocols and historical protocol effectiveness data associated with historical therapeutic protocols are analyzed to determine and select one or more effective therapeutic protocols.

In one embodiment, one or more of the new therapeutic protocols represented by the new therapeutic protocol test data are selected, wherein the new therapeutic protocols have been found to be effective. As discussed above, a determination as to what constitutes an “effective” protocol may be made by setting a threshold value for the effectiveness ratings represented by the predicted therapeutic protocol effectiveness data. In one embodiment, historical protocol effectiveness data may also be considered in determining and selecting effective protocols.

In one embodiment, once one or more effective therapeutic protocols are selected at 516, process flow proceeds to 518. In one embodiment, at 518, the one or more effective therapeutic protocols are utilized to generate one or more maximally effective therapeutic protocols.

In one embodiment, once one or more effective therapeutic protocols have been selected, effective therapeutic protocol definition data is generated, which contains data defining the one or more selected effective protocols. In one embodiment, the effective therapeutic protocol definition data is then used to generate one or more maximally effective therapeutic protocols. In various embodiments, the maximally effective therapeutic protocols may include any number and combination of maximally effective protocols, and each of these protocols or protocol combinations is defined by maximally effective protocol definition data.

In one embodiment, once one or more maximally effective therapeutic protocols are generated at 518, process flow proceeds to 520. In one embodiment, at 520, maximally effective protocol definition data associated with the one or more maximally effective therapeutic protocols is stored as historical protocol definition data for future use in administration of a psychological therapy.

Referring briefly to FIG. 4 and FIG. 5 together, in various embodiments, once one or more maximally effective therapeutic protocols have been generated, the maximally effective protocol definition data representing the maximally effective therapeutic protocols may be stored in a data structure for further use. For example, in one embodiment, maximally effective protocol definition data is stored as historical protocol definition data. This is advantageous because it creates a feedback loop for the machine learning process, wherein the newly generated maximally effective therapeutic protocols can be incorporated into the therapeutic protocol effectiveness model training data, which is generated at 416 of FIG. 4, and is used to train the therapeutic protocol effectiveness prediction models at 418 of FIG. 4. In this manner, the trained therapeutic protocol effectiveness prediction models may be continually updated and refined as new patient protocol response data is received from one or more patients.

In one embodiment, once the maximally effective protocol definition data is stored at 520, process flow proceeds to 522. In one embodiment, at 522, the selected psychological therapy is administered to the patient according to the one or more maximally effective therapeutic protocols.

In one embodiment, once one or more maximally effective therapeutic protocols have been generated and/or stored, the one or more maximally effective therapeutic protocols may be incorporated into a therapy, which may then be administered to a patient, such as the current patient. In some embodiments, once generated, the maximally effective therapeutic protocols may be automatically incorporated into a therapy for administration to the current patient. In some embodiments, a health practitioner may review the maximally effective therapeutic protocols prior to incorporation into a therapy for administration to the current patient. In some embodiments, the one or more maximally effective therapeutic protocols may be incorporated into a therapy, but the therapy may not be administered to the current patient and/or the therapy may be administered to a patient other than the current patient. In various embodiments, the therapy may be administered to the current patient using one or more communication mechanisms. In some embodiments, communication mechanisms include a health practitioner conducting a physical in-person meeting with the current patient to verbally guide the current patient through the therapy. In other embodiments, communication mechanisms include administering the therapy to the current patient remotely, for example through a website, or through one or more software applications that can be executed from computing systems associated with the current patient.

In one embodiment, once the selected psychological therapy is administered to the patient at 522, process flow proceeds to END 524 and the process 500 for utilizing trained therapeutic protocol effectiveness prediction models to generate maximally effective therapeutic protocols for a specific patient is exited to await new data and/or instructions.

FIG. 6 is a flow chart of a process 600 for utilizing trained therapeutic protocol effectiveness prediction models to generate generalized maximally effective therapeutic protocols, in accordance with one embodiment.

Process 600 begins at BEGIN 602 and process flow proceeds to 604. At 604, a psychological therapy is selected for administration to one or more patients.

As noted above, there are a variety of established and/or clinically validated therapies that have been shown to provide benefit to patients, and administration of these clinically validated therapies are typically governed by a collection of therapeutic protocols associated with the particular therapy. In one embodiment, a therapy is selected for future administration to one or more patients.

In one embodiment, once a psychological therapy is selected for administration to one or more patients at 604, process flow proceeds to 606. In one embodiment, at 606, new therapeutic protocol test data is generated representing one or more new therapeutic protocols associated with the psychological therapy.

In various embodiments, new therapeutic protocol test data is generated representing one or more new therapeutic protocols. As noted above, in various embodiments, new therapeutic protocols may also be thought of as potential therapeutic protocols or candidate therapeutic protocols, in the sense that they are protocols that are being considered for use in a therapy. In one embodiment, the new therapeutic protocol test data includes data representing any number of new therapeutic protocols.

In one embodiment, once new therapeutic protocol test data is generated at 606, process flow proceeds to 608. In one embodiment, at 608, the new therapeutic protocol test data is provided to the one or more trained therapeutic protocol effectiveness prediction models.

In one embodiment, once the new therapeutic protocol test data is provided to the one or more trained therapeutic protocol effectiveness prediction models at 608, process flow proceeds to 610. In one embodiment, at 610, the one or more trained therapeutic protocol effectiveness prediction models are utilized to generate predicted protocol effectiveness data for the new protocols represented by the new therapeutic protocol test data. In various embodiments, the predicted therapeutic protocol effectiveness data represents the predicted effectiveness of each of the new therapeutic protocols represented by the new therapeutic protocol test data.

In one embodiment, once predicted protocol effectiveness data is generated at 610, process flow proceeds to 612. In one embodiment, at 612, the predicted protocol effectiveness data associated with the new therapeutic protocols and historical protocol effectiveness data associated with historical therapeutic protocols are analyzed to determine and select one or more effective therapeutic protocols.

In one embodiment, one or more of the new therapeutic protocols represented by the new therapeutic protocol test data are selected, wherein the new therapeutic protocols have been found to be effective. As discussed above, a determination as to what constitutes an “effective” protocol may be made by setting a threshold value for the effectiveness ratings represented by the predicted therapeutic protocol effectiveness data. In one embodiment, historical protocol effectiveness data may also be considered in determining and selecting effective protocols.

In one embodiment, once one or more effective therapeutic protocols are selected at 612, process flow proceeds to 614. In one embodiment, at 614, the one or more effective therapeutic protocols are utilized to generate one or more maximally effective therapeutic protocols.

In one embodiment, once one or more effective therapeutic protocols have been selected, effective therapeutic protocol definition data is generated, which contains data defining the one or more selected effective protocols. In one embodiment, the effective therapeutic protocol definition data is then used to generate one or more maximally effective therapeutic protocols. In various embodiments, the maximally effective therapeutic protocols may include any number and combination of maximally effective protocols, and each of these protocols or protocol combinations is defined by maximally effective protocol definition data.

In one embodiment, once one or more maximally effective therapeutic protocols are generated at 614, process flow proceeds to 616. In one embodiment, at 616, maximally effective protocol definition data associated with the one or more maximally effective therapeutic protocols is stored as historical protocol definition data for future use in administration of a psychological therapy.

Referring briefly to FIG. 4 and FIG. 6 together, in various embodiments, once one or more maximally effective therapeutic protocols have been generated, the maximally effective protocol definition data representing the maximally effective therapeutic protocols may be stored in a data structure for further use. For example, in one embodiment, maximally effective protocol definition data is incorporated into historical protocol definition data. This is advantageous because it creates a feedback loop for the machine learning process, wherein the newly generated maximally effective therapeutic protocols can be incorporated into the therapeutic protocol effectiveness model training data, which is generated at 416 of FIG. 4, and is used to train the therapeutic protocol effectiveness prediction models at 418 of FIG. 4. In this manner, the trained therapeutic protocol effectiveness prediction models may be continually updated and refined as new patient protocol response data is received from one or more patients.

In one embodiment, once the maximally effective protocol definition data is incorporated into historical protocol definition data at 616, process flow proceeds to 618. In one embodiment, at 618, the psychological therapy is administered to the average patient according to the one or more maximally effective therapeutic protocols.

In one embodiment, once one or more maximally effective therapeutic protocols have been generated, the one or more maximally effective therapeutic protocols may be incorporated into a therapy, which may then be administered to an average patient. In some embodiments, once generated, the maximally effective therapeutic protocols may be automatically incorporated into a therapy for administration to the average patient. In some embodiments, a health practitioner may review the maximally effective therapeutic protocols prior to incorporation into the therapy. In some embodiments, the maximally effective therapeutic protocols may be stored in a data structure for further use, but might not be incorporated into a particular therapy. In some embodiments, the one or more maximally effective therapeutic protocols may be incorporated into a therapy, but the therapy may not be administered to an average patient. In various embodiments, the therapy may be administered to the average patient using one or more communication mechanisms. In some embodiments, communication mechanisms include a health practitioner conducting a physical in-person meeting with the average patient to verbally guide the average patient through the therapy. In other embodiments, communication mechanisms include administering the therapy to the average patient remotely, for example through a website, or through one or more software applications that can be executed from computing systems associated with the current patient.

In one embodiment, once the maximally effective protocol definition data is stored at 616, process flow proceeds to END 620 and the process 600 for utilizing trained therapeutic protocol effectiveness prediction models to generate generalized maximally effective therapeutic protocols is exited to await new data and/or instructions.

In one embodiment, a computing system implemented method comprises selecting a psychological therapy for administration to one or more patients, administering the therapy to the one or more patients according to one or more historical therapeutic protocols associated with the selected psychological therapy, monitoring the responses of the one or more patients to the one or more historical therapeutic protocols to obtain patient protocol response data, analyzing the patient protocol response data to determine the effectiveness of the one or more historical therapeutic protocols for the one or more patients, and generating patient protocol effectiveness data representing the effectiveness of the one or more historical therapeutic protocols for the one or more patients. In one embodiment, a computing system implemented method further comprises analyzing the patient protocol effectiveness data and patient data associated with the one or more patients to generate one or more patient profiles. In one embodiment, a computing system implemented method further comprises correlating historical therapeutic protocol data associated with the one or more historical therapeutic protocols with patient profile data associated with the one or more patient profiles to generate therapeutic protocol effectiveness model training data. In one embodiment, a computing system implemented method comprises correlating historical therapeutic protocol data associated with the one or more historical therapeutic protocols with patient protocol effectiveness data associated with the responses of the one or more patients to the one or more historical therapeutic protocols to generate therapeutic protocol effectiveness model training data. In one embodiment, a computing system implemented method further comprises utilizing the therapeutic protocol effectiveness model training data to train one or more therapeutic protocol effectiveness prediction models, thereby resulting in the creation of one or more trained therapeutic protocol effectiveness prediction models.

In one embodiment, a computing system implemented method further comprises selecting the psychological therapy for administration to a current patient, analyzing current patient data associated with the current patient and patient profile data associated with one or more predefined patient profiles to select a patient profile for the current patient, and providing selected patient profile data associated with the selected patient profile to the one or more trained therapeutic protocol effectiveness prediction models. In one embodiment, a computing system implemented method comprises generating new therapeutic protocol test data representing one or more new therapeutic protocols associated with the psychological therapy, and providing the new therapeutic protocol test data to the one or more trained therapeutic protocol effectiveness prediction models. In one embodiment, a computing system implemented method further comprises utilizing the one or more trained therapeutic protocol effectiveness prediction models to generate predicted protocol effectiveness data for the new therapeutic protocols represented by the new therapeutic protocol test data, analyzing the predicted protocol effectiveness data associated with the one or more new therapeutic protocols and historical protocol effectiveness data associated with the one or more historical therapeutic protocols to determine and select one or more effective therapeutic protocols, utilizing effective protocol definition data associated with the one or more effective therapeutic protocols to generate one or more maximally effective therapeutic protocols, and upon generation of one or more maximally effective therapeutic protocols, taking one or more actions.

In one embodiment, taking one or more actions includes one or more of storing maximally effective therapeutic protocol definition data associated with the one or more maximally effective therapeutic protocols for use in administration of a psychological therapy, administering the psychological therapy to one or more patients according to the one or more maximally effective therapeutic protocols, storing maximally effective therapeutic protocol definition data associated with the one or more maximally effective therapeutic protocols for incorporation into the therapeutic protocol effectiveness model training data, and incorporating the one or more maximally effective therapeutic protocols into the therapeutic protocol effectiveness model training data.

In one embodiment, generating one or more maximally effective therapeutic protocols includes replacing one or more of the historical therapeutic protocols with one or more of the effective therapeutic protocols. In one embodiment, the therapy is a psychological therapy. In one embodiment, the psychological therapy is a cognitive behavioral therapy (CBT). In one embodiment, the cognitive behavioral therapy (CBT) is used to treat patients diagnosed with irritable bowel syndrome (IBS). In one embodiment, the therapy is administered remotely. In one embodiment, the responses of the one or more patients to the one or more historical therapeutic protocols are monitored remotely.

In one embodiment, the one or more therapeutic protocol effectiveness prediction models are machine learning based models that are one or more of supervised machine learning-based models, semi supervised machine learning-based models, unsupervised machine learning-based models, classification machine learning-based models, logistical regression machine learning-based models, neural network machine learning-based models, and deep learning machine learning-based models.

In one embodiment, a system comprises one or more processors and one or more physical memories, the one or more physical memories having stored therein data representing instructions which when processed by the one or more processors perform the above described computer implemented method/process.

The above described method and system result in generation of one or more maximally effective therapeutic protocols, which may be incorporated into a therapy for administration to one or more patients, thus ensuring that the patients receive effective care, support, and treatment. Further, the machine learning processes described above employ a feedback loop, such that the one or more therapeutic effectiveness prediction models can be dynamically refined to account for newly received effectiveness data, thus continually improving the accuracy of future effectiveness predictions generated by the model. As a result of these and other disclosed features, discussed in detail above, the disclosed embodiments provide an effective and efficient technical solution to the technical problem of dynamically generating therapeutic protocols to ensure that patients receive maximally effective care, support, and treatment.

Consequently, the embodiments disclosed herein are not an abstract idea, and are well-suited to a wide variety of practical applications. Further, many of the embodiments disclosed herein require processing and analysis of billions of data points and combinations of data points, and thus, the technical solution disclosed herein cannot be implemented solely by mental steps or pen and paper, is not an abstract idea, and is, in fact, directed to providing technical solutions to long-standing technical problems associated with predicting the effectiveness of therapeutic protocols and generating protocols that will be maximally effective when incorporated into a therapy for administration to a patient.

Additionally, the disclosed method and system for dynamically generating therapeutic protocols using machine learning models requires a specific process comprising the aggregation and detailed analysis of large quantities of patient data, protocol data, and protocol effectiveness data, and as such, does not encompass, embody, or preclude other forms of innovation in the area of therapeutic protocol generation. Further, the disclosed embodiments of systems and methods for dynamically generating therapeutic protocols using machine learning models are not abstract ideas for at least several reasons.

First, dynamically generating therapeutic protocols using machine learning models is not an abstract idea because it is not merely an idea in and of itself. For example, the process cannot be performed mentally or using pen and paper, as it is not possible for the human mind to identify, process, and analyze the millions of possible patient characteristics, protocols, combinations of protocols, and associated protocol effectiveness data, even with pen and paper to assist the human mind and even with unlimited time.

Second, dynamically generating therapeutic protocols using machine learning models is not a fundamental economic practice (e.g., is not merely creating a contractual relationship, hedging, mitigating a settlement risk, etc.).

Third, dynamically generating therapeutic protocols using machine learning models is not merely a method of organizing human activity (e.g., managing a game of bingo). Rather, in the disclosed embodiments, the method and system for dynamically generating therapeutic protocols using machine learning models provides a tool that significantly improves the fields of medical and mental health care. Through the disclosed embodiments, health practitioners are provided with a tool to help them generate improved therapeutic protocols, which ensures that patients are provided with personalized and maximally effective assistance, treatment, and care. As such, the method and system disclosed herein is not an abstract idea, and also serves to integrate the ideas disclosed herein into practical applications of those ideas.

Fourth, although mathematics may be used to implement the embodiments disclosed herein, the systems and methods disclosed and claimed herein are not abstract ideas because the disclosed systems and methods are not simply a mathematical relationship/formula.

It should be noted that the language used in the specification has been principally selected for readability, clarity, and instructional purposes, and may not have been selected to delineate or circumscribe the inventive subject matter. Accordingly, the disclosure of the present invention is intended to be illustrative, but not limiting, of the scope of the invention, which is set forth in the claims below.

The present invention has been described in particular detail with respect to specific possible embodiments. Those of skill in the art will appreciate that the invention may be practiced in other embodiments. For example, the nomenclature used for components, capitalization of component designations and terms, the attributes, data structures, or any other programming or structural aspect is not significant, mandatory, or limiting, and the mechanisms that implement the invention or its features can have various different names, formats, or protocols. Further, the system or functionality of the invention may be implemented via various combinations of software and hardware, as described, or entirely in hardware elements. Also, particular divisions of functionality between the various components described herein are merely exemplary, and not mandatory or significant. Consequently, functions performed by a single component may, in other embodiments, be performed by multiple components, and functions performed by multiple components may, in other embodiments, be performed by a single component.

In the discussion above, certain aspects of one embodiment include process steps and/or operations and/or instructions described herein for illustrative purposes in a particular order and/or grouping. However, the particular order and/or grouping shown and discussed herein are illustrative only and not limiting. Those of ordinary skill in the art will recognize that other orders and/or grouping of the process steps and/or operations and/or instructions are possible and, in some embodiments, one or more of the process steps and/or operations and/or instructions discussed above can be combined and/or deleted. In addition, portions of one or more of the process steps and/or operations and/or instructions can be re-grouped as portions of one or more other of the process steps and/or operations and/or instructions discussed herein. Consequently, the particular order and/or grouping of the process steps and/or operations and/or instructions discussed herein do not limit the scope of the invention as claimed below.

As discussed in more detail above, using the above embodiments, with little or no modification and/or input, there is considerable flexibility, adaptability, and opportunity for customization to meet the specific needs of various parties under numerous circumstances.

Some portions of the above description present the features of the present invention in terms of algorithms and symbolic representations of operations, or algorithm-like representations, of operations on information/data. These algorithmic or algorithm-like descriptions and representations are the means used by those of skill in the art to most effectively and efficiently convey the substance of their work to others of skill in the art. These operations, while described functionally or logically, are understood to be implemented by computer programs or computing systems. Furthermore, it has also proven convenient at times to refer to these arrangements of operations as steps or modules or by functional names, without loss of generality.

Unless specifically stated otherwise, as would be apparent from the above discussion, it is appreciated that throughout the above description, discussions utilizing terms such as, but not limited to, “activating”, “accessing”, “adding”, “aggregating”, “alerting”, “applying”, “analyzing”, “associating”, “calculating”, “capturing”, “categorizing”, “classifying”, “comparing”, “creating”, “defining”, “detecting”, “determining”, “distributing”, “eliminating”, “encrypting”, “extracting”, “filtering”, “forwarding”, “generating”, “identifying”, “implementing”, “informing”, “monitoring”, “obtaining”, “posting”, “processing”, “providing”, “receiving”, “requesting”, “saving”, “sending”, “storing”, “substituting”, “transferring”, “transforming”, “transmitting”, “using”, etc., refer to the action and process of a computing system or similar electronic device that manipulates and operates on data represented as physical (electronic) quantities within the computing system memories, resisters, caches or other information storage, transmission or display devices.

The present invention also relates to an apparatus or system for performing the operations described herein. This apparatus or system may be specifically constructed for the required purposes, or the apparatus or system can comprise a system selectively activated or configured/reconfigured by a computer program stored on a non-transitory computer readable medium for carrying out instructions using a processor to execute a process, as discussed or illustrated herein that can be accessed by a computing system or other device.

Those of ordinary skill in the art will readily recognize that the algorithms and operations presented herein are not inherently related to any particular computing system, computer architecture, computer or industry standard, or any other specific apparatus. Various systems may also be used with programs in accordance with the teaching herein, or it may prove more convenient/efficient to construct more specialized apparatuses to perform the required operations described herein. The required structure for a variety of these systems will be apparent to those of ordinary skill in the art, along with equivalent variations. In addition, the present invention is not described with reference to any particular programming language and it is appreciated that a variety of programming languages may be used to implement the teachings of the present invention as described herein, and any references to a specific language or languages are provided for illustrative purposes only and for enablement of the invention as contemplated by the inventors at the time of filing.

The present invention is well suited to a wide variety of computer network systems operating over numerous topologies. Within this field, the configuration and management of large networks comprise storage devices and computers that are communicatively coupled to similar or dissimilar computers and storage devices over a private network, a LAN, a WAN, a private network, or a public network, such as the Internet.

It should also be noted that the language used in the specification has been principally selected for readability, clarity and instructional purposes, and may not have been selected to delineate or circumscribe the inventive subject matter. Accordingly, the disclosure of the present invention is intended to be illustrative, but not limiting, of the scope of the invention, which is set forth in the claims below.

In addition, the operations shown in the figures, or as discussed herein, are identified using a particular nomenclature for ease of description and understanding, but other nomenclature is often used in the art to identify equivalent operations.

Therefore, numerous variations, whether explicitly provided for by the specification or implied by the specification or not, may be implemented by one of skill in the art in view of this disclosure.

What is claimed is:

1. A computing system implemented method comprising:
administering a therapy to one or more patients according to one or more historical therapeutic protocols associated with the therapy;
analyzing patient protocol response data representing the responses of the one or more patients to the one or more historical therapeutic protocols to determine the effectiveness of the one or more historical therapeutic protocols for the one or more patients;
analyzing patient protocol effectiveness data representing the effectiveness of the one or more historical therapeutic protocols for the one or more patients and patient data associated with the one or more patients to generate one or more patient profiles;
correlating historical therapeutic protocol data, the patient protocol effectiveness data, and patient profile data associated with the one or more patient profiles to generate therapeutic protocol effectiveness model training data;
utilizing the therapeutic protocol effectiveness model training data to train one or more therapeutic protocol effectiveness prediction models, thereby resulting in the creation of one or more trained therapeutic protocol effectiveness prediction models; and
utilizing the one or more trained therapeutic protocol effectiveness prediction models to generate one or more maximally effective therapeutic protocols.

2. The method of claim 1 wherein utilizing the one or more trained therapeutic protocol effectiveness prediction models to generate one or more maximally effective therapeutic protocols further includes:
analyzing current patient data associated with a current patient and patient profile data associated with one or more patient profiles to select a patient profile for the current patient; and
providing selected patient profile data associated with the selected patient profile to the one or more trained therapeutic protocol effectiveness prediction models;
generating new therapeutic protocol test data representing one or more new therapeutic protocols associated with the therapy;
providing the new therapeutic protocol test data to the one or more trained therapeutic protocol effectiveness prediction models;
utilizing the one or more trained therapeutic protocol effectiveness prediction models to generate predicted protocol effectiveness data for the new therapeutic protocols represented by the new therapeutic protocol test data;
analyzing the predicted protocol effectiveness data associated with the one or more new therapeutic protocols and historical protocol effectiveness data associated with the one or more historical therapeutic protocols to determine and select one or more effective therapeutic protocols;
utilizing effective therapeutic protocol definition data associated with the one or more effective therapeutic protocols to generate one or more maximally effective therapeutic protocols; and
upon generation of one or more maximally effective therapeutic protocols, taking one or more actions.

3. The method of claim 2 wherein taking one or more actions includes one or more of:
storing maximally effective therapeutic protocol definition data associated with the one or more maximally effective therapeutic protocols for use in administration of a therapy;
administering the therapy to one or more patients according to the one or more maximally effective therapeutic protocols;
storing maximally effective therapeutic protocol definition data associated with the one or more maximally effective therapeutic protocols for incorporation into the therapeutic protocol effectiveness model training data; and
incorporating the one or more maximally effective therapeutic protocols into the therapeutic protocol effectiveness model training data.

4. The method of claim 2 wherein generating one or more maximally effective therapeutic protocols includes replacing one or more of the historical therapeutic protocols with one or more of the effective therapeutic protocols.

5. The method of claim 1 wherein the therapy is a psychological therapy.

6. The method of claim 5 wherein the psychological therapy is a cognitive behavioral therapy (CBT).

7. The method of claim 6 wherein the cognitive behavioral therapy (CBT) is used to treat patients diagnosed with irritable bowel syndrome (IBS).

8. The method of claim 1 wherein the therapy is administered remotely.

9. The method of claim 1 wherein the responses of the one or more patients to the one or more historical therapeutic protocols are monitored remotely.

10. The method of claim 1 wherein the one or more therapeutic protocol effectiveness prediction models are machine learning based models that are one or more of:
supervised machine learning-based models;
semi supervised machine learning-based models;
unsupervised machine learning-based models;
classification machine learning-based models;
logistical regression machine learning-based models;
neural network machine learning-based models; and
deep learning machine learning-based models.

11. A system comprising:
one or more processors; and
one or more physical memories, the one or more physical memories having stored therein data representing instructions which when processed by the one or more processors perform a process, the process comprising:
administering a therapy to one or more patients according to one or more historical therapeutic protocols associated with the therapy;
analyzing patient protocol response data representing the responses of the one or more patients to the one or more historical therapeutic protocols to determine the effectiveness of the one or more historical therapeutic protocols for the one or more patients;
analyzing patient protocol effectiveness data representing the effectiveness of the one or more historical therapeutic protocols for the one or more patients and patient data associated with the one or more patients to generate one or more patient profiles;
correlating historical therapeutic protocol data, the patient protocol effectiveness data, and patient profile data associated with the one or more patient profiles to generate therapeutic protocol effectiveness model training data;
utilizing the therapeutic protocol effectiveness model training data to train one or more therapeutic protocol effectiveness prediction models, thereby resulting in the creation of one or more trained therapeutic protocol effectiveness prediction models; and
utilizing the one or more trained therapeutic protocol effectiveness prediction models to generate one or more maximally effective therapeutic protocols.

12. The system of claim 11 wherein utilizing the one or more trained therapeutic protocol effectiveness prediction models to generate one or more maximally effective therapeutic protocols further includes:
analyzing current patient data associated with a current patient and patient profile data associated with one or more patient profiles to select a patient profile for the current patient; and
providing selected patient profile data associated with the selected patient profile to the one or more trained therapeutic protocol effectiveness prediction models;
generating new therapeutic protocol test data representing one or more new therapeutic protocols associated with the therapy;
providing the new therapeutic protocol test data to the one or more trained therapeutic protocol effectiveness prediction models;
utilizing the one or more trained therapeutic protocol effectiveness prediction models to generate predicted protocol effectiveness data for the new therapeutic protocols represented by the new therapeutic protocol test data;
analyzing the predicted protocol effectiveness data associated with the one or more new therapeutic protocols and historical protocol effectiveness data associated with the one or more historical therapeutic protocols to determine and select one or more effective therapeutic protocols;
utilizing effective therapeutic protocol definition data associated with the one or more effective therapeutic protocols to generate one or more maximally effective therapeutic protocols; and
upon generation of one or more maximally effective therapeutic protocols, taking one or more actions.

13. The system of claim 11 wherein taking one or more actions includes one or more of:
storing maximally effective therapeutic protocol definition data associated with the one or more maximally effective therapeutic protocols for use in administration of a psychological therapy;
administering the psychological therapy to one or more patients according to the one or more maximally effective therapeutic protocols;
storing maximally effective therapeutic protocol definition data associated with the one or more maximally effective therapeutic protocols for incorporation into the therapeutic protocol effectiveness model training data; and
incorporating the one or more maximally effective therapeutic protocols into the therapeutic protocol effectiveness model training data.

14. The system of claim 11 wherein generating one or more maximally effective therapeutic protocols includes replacing one or more of the historical therapeutic protocols with one or more of the effective therapeutic protocols .

15. The system of claim 11 wherein the therapy is a psychological therapy.

16. The system of claim 15 wherein the psychological therapy is a cognitive behavioral therapy (CBT).

17. The system of claim 16 wherein the cognitive behavioral therapy (CBT) is used to treat patients diagnosed with irritable bowel syndrome (IBS).

18. The system of claim 11 wherein the one or more therapeutic protocol effectiveness prediction models are machine learning based models that are one or more of:
supervised machine learning-based models;
semi-supervised machine learning-based models;
unsupervised machine learning-based models;
classification machine learning-based models;
logistical regression machine learning-based models;
neural network machine learning-based models; and
deep learning machine learning-based models.

19. A computing system implemented method comprising:
selecting a psychological therapy for administration to one or more patients;
administering the therapy to the one or more patients according to one or more historical therapeutic protocols associated with the selected psychological therapy;
monitoring the responses of the one or more patients to the one or more historical therapeutic protocols to obtain patient protocol response data;
analyzing the patient protocol response data to determine the effectiveness of the one or more historical therapeutic protocols for the one or more patients;
generating patient protocol effectiveness data representing the effectiveness of the one or more historical therapeutic protocols for the one or more patients;
analyzing the patient protocol effectiveness data and patient data associated with the one or more patients to generate one or more patient profiles;
correlating historical therapeutic protocol data associated with the one or more historical therapeutic protocols with patient profile data associated with the one or more patient profiles to generate therapeutic protocol effectiveness model training data;
utilizing the therapeutic protocol effectiveness model training data to train one or more therapeutic protocol effectiveness prediction models, thereby resulting in the creation of one or more trained therapeutic protocol effectiveness prediction models;
selecting the psychological therapy for administration to a current patient;
analyzing current patient data associated with the current patient and patient profile data associated with one or more predefined patient profiles to select a patient profile for the current patient;
providing selected patient profile data associated with the selected patient profile to the one or more trained therapeutic protocol effectiveness prediction models;
generating new therapeutic protocol test data representing one or more new therapeutic protocols associated with the psychological therapy;
providing the new therapeutic protocol test data to the one or more trained therapeutic protocol effectiveness prediction models;
utilizing the one or more trained therapeutic protocol effectiveness prediction models to generate predicted protocol effectiveness data for the new therapeutic protocols represented by the new therapeutic protocol test data;
analyzing the predicted protocol effectiveness data associated with the one or more new therapeutic protocols and historical protocol effectiveness data associated with the one or more historical therapeutic protocols to determine and select one or more effective therapeutic protocols;
utilizing effective protocol definition data associated with the one or more effective therapeutic protocols to generate one or more maximally effective therapeutic protocols;
upon generation of one or more maximally effective therapeutic protocols, taking one or more actions.

20. The method of claim 19 wherein taking one or more actions includes one or more of:
storing maximally effective therapeutic protocol definition data associated with the one or more maximally effective therapeutic protocols for use in administration of a psychological therapy;
administering the psychological therapy to one or more patients according to the one or more maximally effective therapeutic protocols;
storing maximally effective therapeutic protocol definition data associated with the one or more maximally effective therapeutic protocols for incorporation into the therapeutic protocol effectiveness model training data; and
incorporating the one or more maximally effective therapeutic protocols into the therapeutic protocol effectiveness model training data.
